(19) United States
(12) Patent Application Publication
(10) Pub. No.: US 2015/0112047 A1
(43) Pub. Date: Apr. 23, 2015

(54) ADAPTER MOLECULE CAPABLE OF REVERSIBLY EQUIPPING A FUSION PROTEIN CARRYING AN OLIGOHISTIDINE AFFINITY TAG WITH A FURTHER AFFINITY TAG AND METHODS OF USING THE SAME

(71) Applicant: IBA GMBH, GOETTINGEN (DE)

(72) Inventor: Thomas SCHMIDT, Adelebsen (DE)

(21) Appl. No.: 14/524,000

(22) Filed: Oct. 27, 2014

Related U.S. Application Data
(63) Continuation of application No. PCT/EP2013/058745, filed on Apr. 26, 2013.
(60) Provisional application No. 61/638,680, filed on Apr. 26, 2012.

(30) Foreign Application Priority Data
May 25, 2012 (EP) 12169554.8
Apr. 26, 2013 (EP) PCT/EP2013/058745

(51) Int. Cl. C07K 1/22; C07K 14/00; C07K 1/13

(52) U.S. Cl. CPC C07K 1/22; C07K 1/13; C07K 14/00; C07K 2319/22; C07K 2319/40; C07K 2319/43; C07K 2319/21
USPC 530/395; 530/409

(57) ABSTRACT

Disclosed is a bifunctional adapter molecule comprising two binding moieties A and B, the adapter molecule being capable of reversibly equipping a fusion protein carrying an oligohistidine affinity tag with a further affinity tag, wherein the binding moiety A comprises at least two chelating groups K, wherein each chelating group is capable of binding to a transition metal ion, thereby rendering moiety A capable of binding to an oligohistidine affinity tag, and the binding moiety B is an affinity tag other than an oligohistidine tag.

### CROSS-REFERENCE TO RELATED APPLICATIONS

This application is a continuation of International Application No. PCT/EP2013/058745, filed Apr. 26, 2013, which claims the benefit of priority to U.S. Provisional Application No. 61/638,680 filed Apr. 26, 2012 and to European Patent Application No. 12169554.8 filed May 25, 2012, each of which is hereby incorporated in its entirety including all tables, figures, and claims.

### FIELD OF THE INVENTION

The present invention relates to a bifunctional adapter molecule comprising two binding moieties A and B, the adapter molecule being capable of reversibly equipping a fusion protein carrying an oligohistidine affinity tag with a further affinity tag. The invention also relates to a method of equipping a fusion protein carrying an oligohistidine affinity tag with a further reversible affinity tag and uses thereof.

### BACKGROUND OF THE INVENTION

Oligohistidine tags (which consist of usually 5 to 10 consecutive imidazole residues with the hexahistine tag (his6-tag) being the most frequently used version) are currently the most frequently used affinity tags for the generic purification of fused proteins (target proteins). These uses are enabled through binding of the oligohistidine tags to complexed transition metal ions. The transition metal ions are first complexed by chelators such as iminodiacetic acid (IDA) or nitrilotriacetic acid (NTA) that are immobilized to a resin (e.g. Sepharose, Superflow, Macroprep). Chelation provides high affinity immobilization under preservation of free binding sites on the transition metal ions that are then still capable to bind the histidine residues of the oligohistidine tag. Since the binding affinity of the oligohistidine tag to, e.g., a single chelated nickel ion is in the range of 10 μM, simultaneous interaction of one oligohistidine tag with several immobilized metal ions is necessary for efficient binding of the target protein (Lata et al., 2005). This is achieved by providing resins loaded at high density with transition metal ions. This purification technology is widely known as immobilized metal ion adsorption chromatography (IMAC) or as metal chelate affinity chromatography. The binding strength may be principally modulated by the metal ion and pH of the buffers. The bound protein can be eluted by competitive elution with, for example, free imidazole, or by lowering pH. Strong chelating agents, such as EDTA, can also be used. The metal ions most frequently used for purification optimization are Zn²⁺, Ni²⁺, Co²⁺, Ca²⁺, Cu²⁺, and Fe³⁺.

The most prominent disadvantage of IMAC is that the resin modified at high density with transition metal ions binds also proteins other than the target protein, i.e. contaminations, which finally results in impure target protein preparations. These contaminations may predominantly bind due to i) ionic interactions or due to ii) a comparatively high content of histidine and/or cysteine and/or tryptophan and/or acidic residues (see, e.g., Wülfing et al., J. Biol. Chem. 2005, 269, 2895-2901).

The ion exchange effect (i) may be reduced through the use of buffers at elevated ionic strength. However, such non-physiologic elevated ionic strength may, to some degree, be detrimental to the target protein thereby leading to target protein preparations of reduced activity.

The other source of non-specific binding (ii) is more delicate to manage as it is based on the same type of interaction with the resin, i.e. coordination of metal ions to amino acid side chains. Fortunately, affinity of said proteinacous contaminations for the metal ion loaded resin is usually lower. Therefore, a strategy to solve this problem of low specificity is the addition of low amounts of imidazole (1-20 mM) to the crude extract to compete with the proteinacous histidine and/or cysteine and/or tryptophan and/or acidic residues of the contaminations for binding to the metal ions, thereby keeping said contaminations away from binding while the target protein is still able to bind. The downside is, however, reduced affinity of the target protein under such competitive conditions for the resin which may result in reduced yield. A complicating issue in that respect is that the effect of affinity reduction on the purification result cannot be predicted and varies from target protein to target protein because the initial affinity of the oligohistidine tag for the chelated metal ions depends on the given target protein (e.g. because of the given steric accessibility of the terminus fused to the oligohistidine tag). It is therefore necessary to determine the optimal imidazole content separately for each target protein for finding optimal discrimination between target protein and contaminations, i.e. to maximally inhibit the binding of the contaminations without too much reducing the yield of the target protein. But it is not always possible to find a satisfying compromise to meet the yield and purity requirements for a given target protein.

A further disadvantage of IMAC is the possibility of metal ion catalyzed oxidation of target proteins, predominantly at exposed cysteine residues which often play a key role for the activity of the target protein. This problem is due to contacting the target protein with a resin loaded at high density with metal ions where reactive contacts with said cysteine residues and dissolved oxygen very likely arise or where leaching metal ions are comparatively abundant due to a highly charged resin thereby freely accessing sensitive sites on the bound target protein for catalyzing oxidation with dissolved oxygen (Riley, The Scientist 2005, Volume 19, Issue 7; Ramage et al., Life Science News 2002, 11, 18-20).

Summarizing, the three predominant weaknesses inherent to IMAC are i) the ion exchange effect, ii) comparatively low specificity and iii) metal ion catalyzed oxidation.

Recently several approaches for the synthesis and use of bis-, tris-, and tetrakis-NTA:metal ion compounds for the non-covalent labeling of oligohistidine tag fusion proteins (target proteins) in solution with fluorescent dyes or biotin have been described (Lata et al., J. Am. Chem. Soc. 2005, 127, 10205-10215; Huang et al., Bioconjugate Chem. 2006, 17, 1592-1600). These compounds interact more specifically than resins charged at high density with transition metal ions with a target protein fused oligohistidine tag for several reasons. Said compounds are i) in solution, ii) used in stochiometric amounts, iii) have a small size and iv) have limited metal ion binding moieties and thereby limited valences for interacting non-specifically with protein derived amino acid residues that are nevertheless sufficient for high affinity oligohistidine tag binding. Non-specific binding to other proteins (contaminations) is only possible if such other proteins display histidine and/or cysteine and/or tryptophan and/or acidic residues at high density and moreover in a spatial configuration appropriate for simultaneously binding to the metal ions of said comparatively small bis-, tris- and/or tetrakis-NTA compounds. In contrast thereto, a resin modified at high density with metal ions is less selective in accommodating histidine and/or cysteine and/or tryptophan and/or acidic residues of the contaminations because these residues need not be presented at high density and in a special spatial configuration. The same argumentation is valid to explain why metal ion loaded bis-, tris- and/or tetrakis-NTA compounds exhibit reduced non-specific binding via ionic interactions. Last but not least, also metal ion catalyzed oxidation is less likely to occur as the metal ion loaded bis-, tris- and/or tetrakis-NTA compounds are used in stochiometric concentrations relative to the oligohistidine tag fused target protein. Stable binding to the oligohistidine tag prevents the chelated metal ions from interacting with other residues on the target protein thereby preventing catalysation of unwanted oxidation processes at distant cysteine residues. Furthermore, the limited use of metal ions per target protein reduces the amount of potentially free metal ions that also might lead to metal ion catalyzed oxidation. Thus, target proteins may be bound in solution by these reagents in a much more flexible and specific fashion under avoidance of the negative side effects observed during IMAC. Thus said reagents might be the basis for the purification of oligohistidine tag fusion proteins under avoidance of the drawbacks of IMAC. As oligohistidine tag fusion proteins prevail in the world of recombinant proteins, alternative and/or complementing methods for improved purification results as compared to IMAC will be of great benefit of the community using oligohistidine tag fusion proteins.

### SUMMARY OF INVENTION

The present invention is based on the unexpected finding that the disadvantages associated with the purification of recombinant oligohistidine tag fusion proteins (target proteins) can be overcome by making such proteins amenable to other, more selective affinity tag purification technologies. For so doing, the present invention provides a bifunctional adapter molecule comprising two binding moieties A and B, the adapter molecule being capable of reversibly equipping a fusion protein carrying an oligohistidine affinity tag with a further affinity tag. The binding moiety A comprises at least two chelating groups K, wherein each chelating group is capable of binding to a transition metal ion, thereby rendering moiety A capable of sufficiently binding to an oligohistidine affinity tag. The binding moiety B is an affinity tag other than an oligohistidine tag. The term “bifunctional” as used herein thus refers to the ability of the adapter reagent to bind both to an oligohistidine affinity tag and the binding moiety B.

The invention also provides a method for equipping a fusion protein carrying an oligohistidine affinity tag with a further reversibly binding affinity tag. The method comprises contacting the target protein carrying an oligohistidine affinity tag with a bifunctional adapter molecule as described herein, and allowing forming a complex between the adaptor molecule and the oligohistidine affinity tag. Such complex may, e.g., be purified on an affinity resin addressed by moiety B of the adapter molecule.

The invention also provides a method of improving the solubility or folding efficiency of a protein of interest, the protein of interest carrying an oligohistidine affinity tag, wherein the method comprises

contacting the protein of interest an oligohistidine affinity tag with a bifunctional adapter molecule comprising two binding moieties A and B, wherein

the binding moiety A of the adapter molecule comprises at least two chelating groups K, wherein each chelating group is capable of binding to a transition metal ion, thereby rendering moiety A capable of binding to an oligohistidine affinity tag, and

the binding moiety B is an peptide based affinity tag other than an oligohistidine tag,

and allowing forming a complex between the adaptor molecule and the oligohistidine affinity tag.

### BRIEF DESCRIPTION OF THE DRAWINGS

The invention will be better understood with reference to the detailed description when considered in conjunction with the non-limiting examples and the accompanying drawings, in which:

FIG. 1 shows the interaction between a chelating group K of the adapter molecule of the invention and two histidine residues of an oligohistine tag via a complexed transition metal ion. In FIG. 1 nitrilotriacetic acid (NTA) is illustratively shown as one out of at least two chelating groups K of moiety A of the adapter molecule. The NTA group binds, in this case via a Ni²⁺ ion, two histidine side chains of an oligohistidine tag fused to a recombinant protein/fusion protein (not shown). By comprising of at least two of such metal ion complexed chelating groups K binding to the oligohistidine tag, the adapter molecule stably but reversibly equips the fusion protein of interest with the second affinity tag that is provided by moiety B of the adapter molecule (not shown).

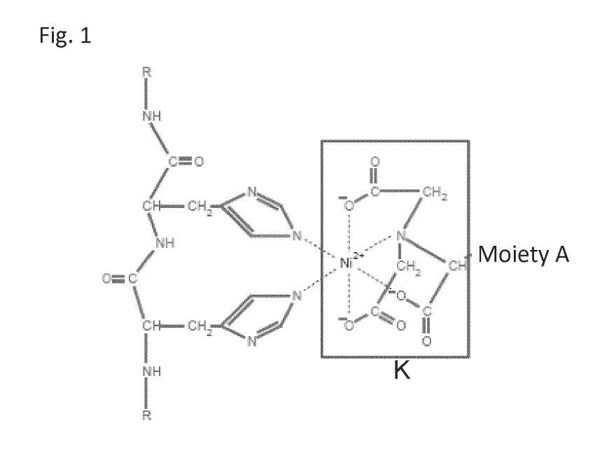

FIG. 2a shows an illustrative example of an adaptor molecule of the present invention. Moiety A comprises three chelating groups K (NTA in this example) which are covalently connected via a shared cyclic scaffold (cyclam, 1,4,8,11-tetraazacyclotetradecane), thereby forming a tris-NTA group. The synthesis of such cyclic tris-NTA-scaffold is described by Lata et al., supra, and can start from compound (1) of international patent application WO 2011/101445. The cyclam scaffold comprises, covalently attached to the 4^{th} N atom of the ring, a linker moiety that connects moiety A to moiety B. In the illustrative compound of FIG. 2a, a linker moiety L1a connects moiety A through an amide bond with moiety B. The amide bond can be formed, for example, by a free primary amino group (see also the Example Section below) reacting with an activated carboxyl group of moiety B, for example, the free C-terminus of the affinity peptide, or an activated side chain group or an artificially introduced carboxyl group. Schematically shown as “X” are histidine side chains of an oligohistidine tag of a fusion protein to which moiety A binds via its metal ion loaded chelating groups K (NTA in this example). Moiety B can be any peptide or carbohydrate based affinity tag, for example a streptavidin binding peptide or an epitope tag such as the myc or the FLAG tag. The adapter molecule may further comprise a label that is bound to moiety A or B (moiety B in the actual example).

FIG. 2b shows two different illustrative examples of moieties A linked to a certain moiety B. The left hand example comprises two chelating groups K (NTA in this example) which are covalently connected via a linear linker, thereby forming a bis-NTA group. The synthesis of such bis-NTA moiety A is described by Lata et al., supra. The right hand moiety A is identical to moiety A of FIG. 2a. FIG. 2b illustrates that the chelating groups K may be connected by completely different linkers to form a moiety A. The two moieties A of FIG. 2b are connected via the same linker L1b to moiety B which is different to the linker L1a of FIG. 2a. This is shown to illustrate that the way of coupling moiety A to moiety B may be realized in many different ways that are all well known to the chemist skilled in the art.

FIG. 3a shows another illustrative example of an adapter molecule of the invention. In the molecule of FIG. 3a, moiety A consists of or comprises three chelating groups K each of which is covalently linked (via the respective sulfur (S)-atom) to the side chain of a cysteine residue (the cysteine residues are also part of moiety A), either directly or via an optionally present internal linker L2 (“internal means that the each of the linker L2 is arranged between atoms/groups within the moiety A). It is possible that between each of the cysteine residues a peptidic linker of any amino acid (Xaa) of a length of, e.g., up to 4 amino acids is present (n and o may have independently from each other a value of 0, 1, 2, 3, or 4). In addition, a linker (Xaa)ₘ of, e.g., up to 50 amino acids between moiety B and moiety A may be present (m may have any value between 1 and 5, or 1 and 10, or 1 and 20, or 1 and 30, or 1 and 40, or 1 and 50. Likewise, the terminal Cys-residue may be connected to an peptide/linker sequence (Xaa)_{q} of e.g., also up to 50 amino acids (q may have any value between 1 and 5, or 1 and 10, or 1 and 20, or 1 and 30, or 1 and 40, or 1 and 50). This linker sequence (Xaa)_{q} might be used to couple a label such as a fluorescent label or a chromophoric label to moiety A.

FIG. 3b shows maleimido-C3-NTA (N-(5-(3-Maleimidopropylamido)-1-carboxy-pentyl)iminodiacetic acid, disodium salt, monohydrate), an illustrative example for an NTA group comprising a linker that carries a maleimide activated group that is able to react with thiol groups of, e.g., cysteine side chains.

FIG. 3c shows an illustrative example of the generic Cys-L2-K unit (entity) of moiety A of FIG. 3a that results after reacting cysteine containing peptides with maleimido-C3-NTA.

FIG. 4 shows another illustrative example of an adapter molecule of the invention. In the illustrative molecule of FIG. 4, the moiety A is directly (without a linker) covalently attached to the C-terminus of a streptavidin binding peptide (Trp-Ser-His-Pro-Gln-Phe-Glu-Lys) representing moiety B. Moiety A comprises three cysteine residues that are coupled to moiety B by conventional peptide chemistry. The thiol groups are coupled to chelating groups K (NTA in this example) via activated internal linkers L3a, L3b and L3c (a, b, c may be identical or different), thereby forming a tris-NTA moiety A. If, e.g., only two cysteine residues were initially coupled to moiety B, a bis-NTA moiety A could have been alternatively formed as well. Similarily, moieties A comprising chelating groups at higher valencies than three can be synthesized easily, simply by coupling more than three cysteine residues (for example, 4, 5 or 6) to moiety B and modifying these cysteine residues with activated, e.g. maleimide-activated, chelating groups, e.g., NTA. Thereby, FIG. 4 illustrates a simple way to synthesize a moiety A specifically binding to oligohistidine tag fusion proteins by using simple peptide chemistry to connect cysteine residues to moiety B that, in a second step, may be easily equipped with chelating groups, e.g. NTA, via conventional maleimide/thiol or other coupling chemistry. The internal linker L3 (“internal” means that the linker L3 is arranged between atoms/groups within the moiety A) may comprise any suitable number of main chain atoms, for example 1 to 20 main chain atoms, wherein the main chain of the internal linker L3 may either contain only carbon atoms or also one or more heteroatoms such as N, O, or S. In some embodiments, the internal linker L3 has 1, 2, 3, 4, 5, 6, 7, 8, 9, 10, 11, 12, 13, 14, 15, 16, 17, 18, 19 or 20 main chain atoms. The cysteine residues are also part of moiety A, with the “first” cysteine residue being linked to the C-terminus of the streptavidin binding peptide, the moiety B. Thus, the adapter molecule of FIG. 4 does not comprise a linker between moiety A and moiety B, even though it is of course possible to insert amino acids between the C-terminal lysine residue of the streptavidin binding peptide (moiety B) and the first cysteine of moiety A.

FIG. 5 shows yet another illustrative example of an adapter molecule of the invention. In the molecule of FIG. 5, moiety A is covalently attached (without a linker) to the C-terminus of a streptavidin binding peptide (three letter code: Trp-Ser-His-Pro-Gln-Phe-Glu-Lys or one letter code: WSHPQFEK, moiety B). Multiple NTA groups (three in this example) are covalently coupled as a whole to the side chain of a single cysteine residue to provide the at least two chelating groups K of moiety A of the adapter molecule. The tris-NTA group in the molecule of FIG. 5 forms (via a first linker) a thioether bond with the side chain of the cysteine and each of the three NTA has a further internal spacer/linker L4a, L4b and L4c, respectively with, for example, each 1 to 6 main chain carbon atoms (C1 to C6). The cysteine residue is also part of moiety A. Thus, the adapter molecule of FIG. 5 does not comprise a linker L between the moieties A and B, even though it is of course also possible to insert amino acids between the C-terminal lysine residue of the streptavidin binding peptide and the cysteine.

FIGS. 6a-6e show different Agilent 2100 Bioanalyzer analyses using an Agilent Protein 230 Kit according to the manufacturer's instructions of different samples of the experiment described below comparing the purification of green fluorescent protein (GFP), C-terminally fused to a hexahistidine tag, from a bacterial crude lysate using (1) an adapter reagent of the invention and Strep-Tactin® affinity chromatography under physiological conditions (PBS pH8), (2) IMAC under same physiological conditions (PBS pH8), and (3) IMAC under non-physiological conditions but under conditions optimized for high selectivity as recommended by the manufacturer Qiagen. Each analysis shows on top, left side, an electropherogram of the protein content of the respective sample, the different proteins being stained by a fluorescent dye and separated under denaturing conditions according to molecular size via capillary electrophoresis. Each peak represents one or more proteins of similar size. On top, right side, the electropherogram is translated into a SDS gel like representation of the separation result. On the bottom of each Figure, the determined size of each protein (mixture) present in a certain peak including its concentration in the sample and its amount relative to the total protein content of the sample representing its degree of purity is shown. FIG. 6a shows the analysis result of the crude lysate after dialysis against PBS pH8 while

FIG. 6b shows the analysis result of the crude lysate in Ni-NTA Lysis Buffer not being dialyzed against PBS pH8. It can be deduced that the protein content of both extracts being the origin of the compared purification results were very similar.

FIG. 6c shows the protein content of the pooled eluates E3 and E4 after Strep-Tactin® affinity purification under physiological conditions (PBS pH8) of the crude lysate containing GFP C-terminally fused to a hexahistidine tag with added adapter reagent as described below.

FIG. 6d shows the protein content of the pooled eluates E3 and E4 after IMAC purification under the same physiological conditions (PBS pH8) of the crude lysate containing GFP C-terminally fused to a hexahistidine tag without having added an adapter reagent of the invention.

FIG. 6e shows the protein content of the pooled eluates E3 and E4 after IMAC purification under non-physiological conditions but under conditions optimal for high selectivity during IMAC (Ni-NTA Lysis and Wash Buffer) of the crude lysate containing GFP C-terminally fused to a hexahistidine tag. When comparing the result of FIGS. 6c and 6d the clear advantage of the purification using the methods of the invention for affinity purification of a hexahistidine tag target protein under physiological conditions versus IMAC is demonstrated as the target protein (migrating @ 27.6 kDa in this case) is enriched to 97.6% purity in comparison of the enrichment of the target protein (migrating @ 27.7 kDa in this case) to 34.6% purity when using IMAC under the same physiological conditions. And even a clear purity advantage becomes apparent when comparing the result of using the methods of the invention under physiological conditions versus IMAC under non-physiological conditions but conditions optimized for high selectivity. Although IMAC provides under the latter conditions the target protein at much higher purity than under physiological conditions, the degree of purity of using the methods of the invention is not reached using conventional IMAC. IMAC under optimal but non-physiological conditions provides the target protein (migrating @ 27.3 kDa in this case) at a purity of 93.3% which means that 6.7% of the protein content are impurities. This means that even after purification of the target protein under non-physiological IMAC buffer conditions that are optimal for high purity, 180% more impurities are included in the target protein preparation versus the impurities (2.4%) present in the target protein preparation purified according to the exemplary method of the invention which were even physiological and thus milder to obtain the target protein at higher activity. The purity result using this exemplary method of the invention is similar to what is expected after conventional Strep-Tactin® affinity chromatography of a target protein containing a directly fused Strep-tag affinity tag. Thus, this further demonstrates that the trisNTA reagents of the invention interact in solution much more specifically with the oligohistidine tag fusion protein than a Ni-NTA affinity resin solid phase loaded with the transition metal ions at high density thereby making clear that the oligohistidine tag:adapter reagent interaction is not a bottleneck for the usefulness of the reagents and methods of the invention. Thus, this adapter approach is of general benefit as the benefits of any affinity tags other than the oligohistidine tag may be brought to an oligohistidine tag fusion protein without being hampered by the oligohistidine tag:adapter reagent interaction.

### DETAILED DESCRIPTION OF THE INVENTION

The invention provides an adapter molecule (or reagent) that comprises an entity A providing at least two chelating groups, e.g., without limitation, compounds published by Lata et al., 2005, supra, or by Huang et al., 2006, supra, for specific binding to an oligohistidine tag fused to a target protein. The adapter molecule comprises at the same time a moiety/entity that is an affinity tag. Such an affinity tag is a first partner of a binding pair such as, without limitation, a streptavidin binding peptide such as a -Trp-Xaa-His-Pro-Gln-Phe-Yaa-Zaa- (SEQ ID NO: 1), wherein Xaa is any amino acid and Yaa and Zaa are both Gly or Yaa is Glu and Zaa is Lys or Arg (formula III), -Trp-Arg-His-Pro-Gln-Phe-Gly-Gly- (formula IV, SEQ ID NO: 2), or c) -Trp-Ser-His-Pro-Gln-Phe-Glu-Lys- (also known as Strep-Tag®) or the SBP-Tag® (sequence: MDEKTTGWRGGHVVEGLAGELEQLRARLEHHPQGQREP, SEQ ID NO: 19), protein A, protein G, protein L, maltose binding protein (MBP), glutathione-S-transferase, glutathione, a calmodulin binding peptide such as CBP® (sequence: KRRWKKNFIAVSAANRFKKISSSGAL (SEQ ID NO: 20), a chitin binding domain such as TNPGVSAWQVNTAYTAGQLVTYNGKTYKCLQPHTSLAGWEPSNVPALWQLQ (SEQ ID NO: 21, a cellulose binding domain, the S-tag (sequence: KETAAAKFERQHMDS, SEQ ID NO: 18) or an affinity peptide (epitope tag, i.e., peptides recognized by an antibody or antibody fragment). Illustrative examples of suitable epitope tags include the FLAG-Tag® (sequence: DYKDDDDK), the Myc-tag (sequence: EQKLISEEDL), the HA-tag (sequence: YPYDVPDYA), the VSV-G-tag (sequence: YTDIEMNRLGK), the HSV-tag (sequence: QPELAPEDPED), or the V5-tag (sequence: GKPIPNPLLGLDST), to mention only a few. The binding moiety B can also be a carbohydrate based affinity tag. Illustrative examples of such carbohydrate based affinity tags include or consist of, but are not limited to, maltose, cellulose or chitin, to name only a few. All of the above mentioned affinity tags (including the epitope tags and the carbohydrate based) tags bind reversibly but specifically bind to a second partner (the affinity tag receptor) of said binding pair. This second partner can, for example, be streptavidin or a streptavidin binding mutein such as Strep-Tactin® in case of a streptavidin binding peptide, an antibody Fc fragment in case of protein A, protein G, or protein L, maltose in case of maltose binding protein, glutathione in case of glutathione-S-transferase (and vice versa in case of glutathione-S-transferase being used as affinity tag), calmodulin in case of a calmodulin binding peptide, chitin in case of a chitin binding domain, cellulose in case of a cellulose binding domain or the S fragment of RNase A in case of the S-tag. In case of an epitope tag such as the FLAG tag or the myc tag (these two epitope tags are purely mentioned for illustrative purposes, the same principle applies to all other epitope tags) the binding partner is an antibody or an antibody fragment thereof such as the anti-FLAG antibody M1 or the anti-myc antibody 9 E10 (cf. Schiweck et al. (1997) FEBS Lett. 414, 33-38). Small adapter reagents are preferred in some embodiments. What becomes clear from these illustrative examples is that all affinity tag technologies that give better results in affinity purification than IMAC, i.e., without limitation, target protein preparations of higher purity and/or activity, or that do not need higher salt concentrations or imidazole or that are not known to catalyze oxidation processes can be addressed by the adapters of the invention by combining such known affinity tags with at least two metal ion chelating groups. It also becomes clear from these illustrative examples that it possible that either partner of the binding pair forms part of the adapter reagent and the other partner is used for immobilization of the adapter reagent. For example, the adapter reagent may comprise or consist of maltose as binding moiety B and then maltose binding protein is used for immobilization of the adapter reagent. Alternatively, the adapter molecule may comprise or consist of maltose binding protein as binding moiety B and then maltose is used for immobilization of the adapter reagent. Preferred are, in some embodiments, adapter molecules comprising peptide based affinity tags as moiety B, wherein peptide means sequences of more than three amino acids.

The adapter molecule can have virtually any structure as long as the molecule comprises a moiety A and a moiety B as defined herein as long as moiety A comprises at least two chelating groups K that are able to (specifically) bind to an oligohistidine tag fused to a target protein and as long as moiety B can act as a further affinity tag.

For example, in one embodiment of an adapter molecule of the invention, the at least two chelating groups K of the binding moiety A may be attached to different locations within the (peptide based or carbohydrate based) affinity tag such that the at least two chelating groups are capable of binding to the same oligohistidine affinity tag. An illustrative example of such an adapter molecule may be a streptavidin binding peptide with the amino acid sequence -Trp-Ser-His-Pro-Gln-Phe-Glu-Lys-(Xaa)n-Trp-Ser-His-Pro-Gln-Phe-Glu-Lys- wherein Xaa is any amino acid and n is an integer from 0 to 12, and in which one or more of the amino acids Xaa carry the at least two chelating groups K. A more concrete example, the adapter molecule may have the sequence -Trp-Ser-His-Pro-Gln-Phe-Glu-Lys-Gly-Gly-Gly-Ser-Cys-(C₁-Cₙ₁-NTA)-Cys-(C₁-Cₙ₂-NTA)-Cys-(C₁-Cₙ₃-NTA)-Gly-Gly-Gly-Ser-Trp-Ser-His-Pro-Gln-Phe-Glu-Lys- wherein each of n1, n2 and n3 has a value independently selected from 0, 1, 2, 3, 4, 5, 6, 7, 8, 9, 10, 11, 12, 13, 14, 15, 16, 17, 18, 19, or 20, and wherein main chain carbon atoms of each of the C₁-Cₙ₁, C₁-C₂ or C₁-C₃ groups are optionally substituted by one or more heteroatoms selected from the group consisting of N, O and S. In this molecule the entire affinity peptide, which is a sequential arrangement of two streptavidin binding modules as described in International Patent Publication WO 02/077018 or U.S. Pat. No. 7,981,632 is moiety B and the Cys residues in the linker between the two streptavidin binding modules that carry the NTA chelating groups represent moiety A. Thus, this arrangement is such that the at least two chelating groups K of the binding moiety A are attached to different locations within the affinity tag such that the at least two chelating groups are capable of binding to the same oligohistidine affinity tag. Another example of such a molecule is glutathione-S-transferase which has attached to surface exposed residues that are spatially in proximity to each other at least two chelating groups K that are able to bind the same oligohistidine affinity tag.

In some embodiments of the adapter molecule that contains a streptavidin binding peptide as moiety B, the streptavidin binding peptide comprises or consists of one of the following sequences:
a) -Trp-Xaa-His-Pro-Gln-Phe-Yaa-Zaa- (SEQ ID NO: 1), wherein Xaa is any amino acid and Yaa and Zaa are both Gly or Yaa is Glu and Zaa is Lys or Arg (formula III),
b) -Trp-Arg-His-Pro-Gln-Phe-Gly-Gly- (formula IV, SEQ ID NO: 2),
c) -Trp-Ser-His-Pro-Gln-Phe-Glu-Lys- (formula V, SEQ ID NO: 3),
d) a sequential arrangement of at least two streptavidin binding peptides, wherein each peptide binds streptavidin, wherein the distance between two peptides is at least 0 and not greater than 50 amino acids and wherein each of the at least two peptides comprises the amino acid sequence -His-Pro-Baa- in which Baa is selected from the group consisting of glutamine, asparagine and methionine (formula VI),
e) a sequential arrangement as recited in d), wherein one of the at least two peptides comprises the sequence -His-Pro-Gln-,
f) a sequential arrangement as recited in d), wherein one of the peptides comprises an amino acid sequence -His-Pro-Gln-Phe- (SEQ ID NO: 4),
g) a sequential arrangement as recited in d) wherein at least one peptide includes at least the amino sequence -Oaa-Xaa-His-Pro-Gln-Phe-Yaa-Zaa- (SEQ ID NO: 5), where Oaa is Trp, Lys or Arg, Xaa is any amino acid and where either Yaa and Zaa are both Gly or Yaa is Glu and Zaa is Lys or Arg,
h) a sequential arrangement as recited in d) wherein at least one peptide includes at least the amino acid sequence -Trp-Xaa-His-Pro-Gln-Phe-Yaa-Zaa- (SEQ ID NO: 6) where Xaa is any amino acid and where either Yaa and Zaa are both Gly or Yaa is Glu and Zaa is Lys or Arg,
i) a sequential arrangement as recited in d) wherein at least one peptide includes at least the amino acid sequence -Trp-Ser-His-Pro-Gln-Phe-Glu-Lys- (SEQ ID NO: 7),
j) the amino acid sequence -Trp-Ser-His-Pro-Gln-Phe-Glu-Lys-(Xaa)n-Trp-Ser-His-Pro-Gln-Phe-Glu-Lys-(SEQ ID NO: 8) wherein Xaa is any amino acid and n is an integer from 0 to 12,
k) -Trp-Ser-His-Pro-Gln-Phe-Glu-Lys-(Xaa)n-Cys-Cys-Cys-(Xaa)n-Trp-Ser-His-Pro-Gln-Phe-Glu-Lys-, (SEQ ID NO: 9) wherein Xaa is any amino acid and n is an integer from 0 to 10,
l) the amino acid sequence -Trp-Ser-His-Pro-Gln-Phe-Glu-Lys-(GlyGlyGlySer)n-Trp-Ser-His-Pro-Gln-Phe-Glu-Lys- (SEQ ID NO: 10), where n is either 2 or 3,
m) the amino acid sequence MDEKTTGWRGGHVVEGLAGELEQLRARLEHHPQGQREP (SEQ ID NO: 19).

The adapter molecule may have a general formula selected from the formulae

B-L-A (I),

or

A-L-B (II),

wherein L is an optionally present linker moiety.

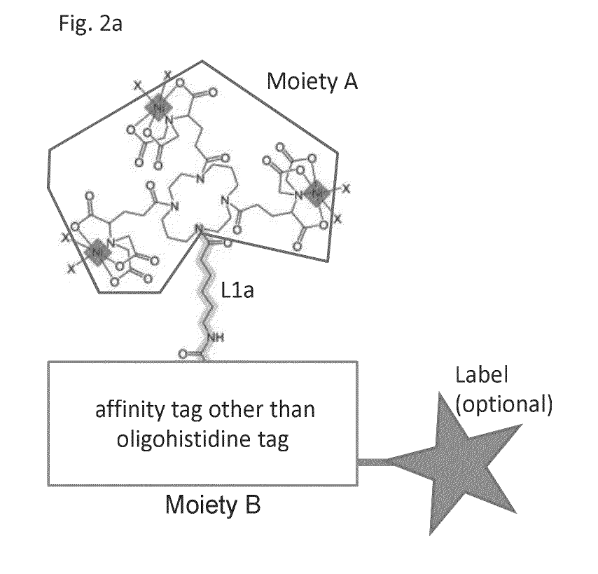

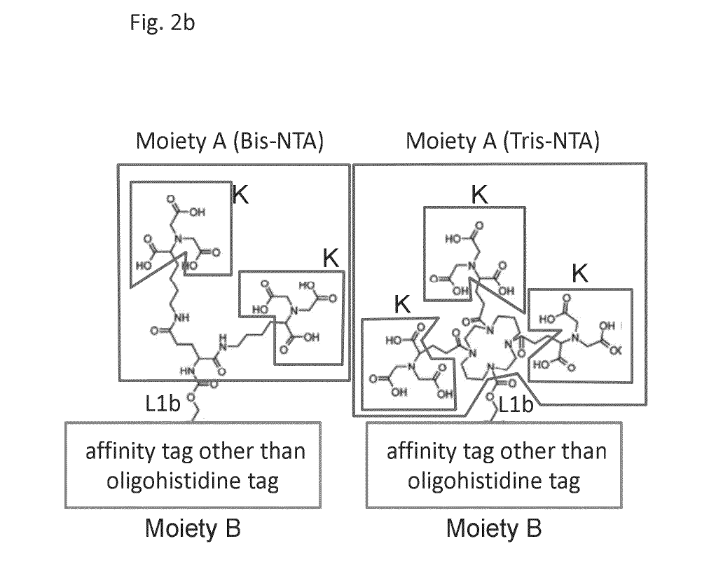

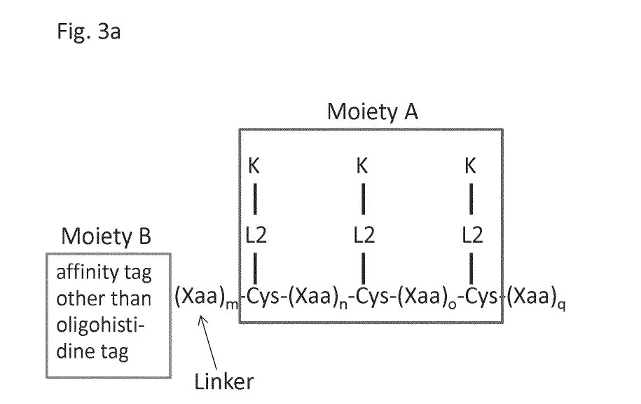

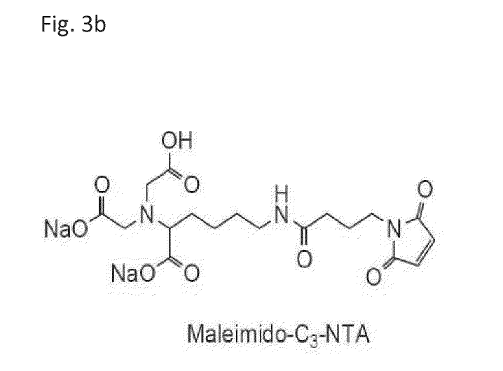

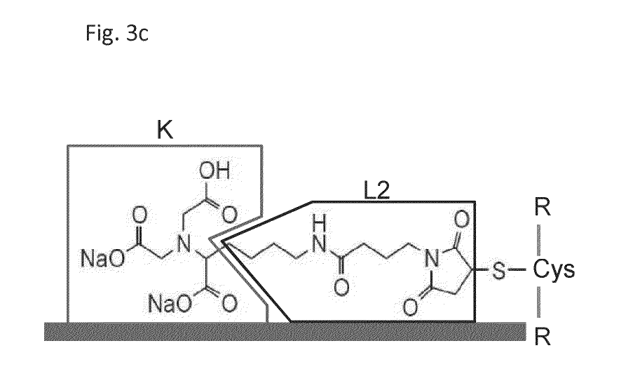

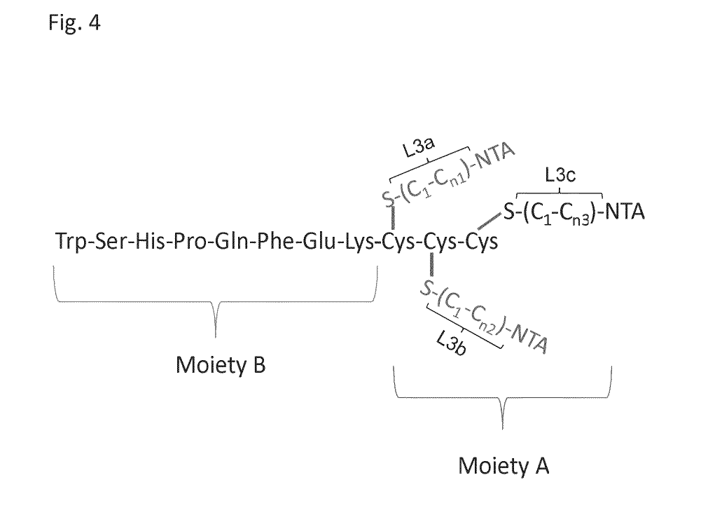

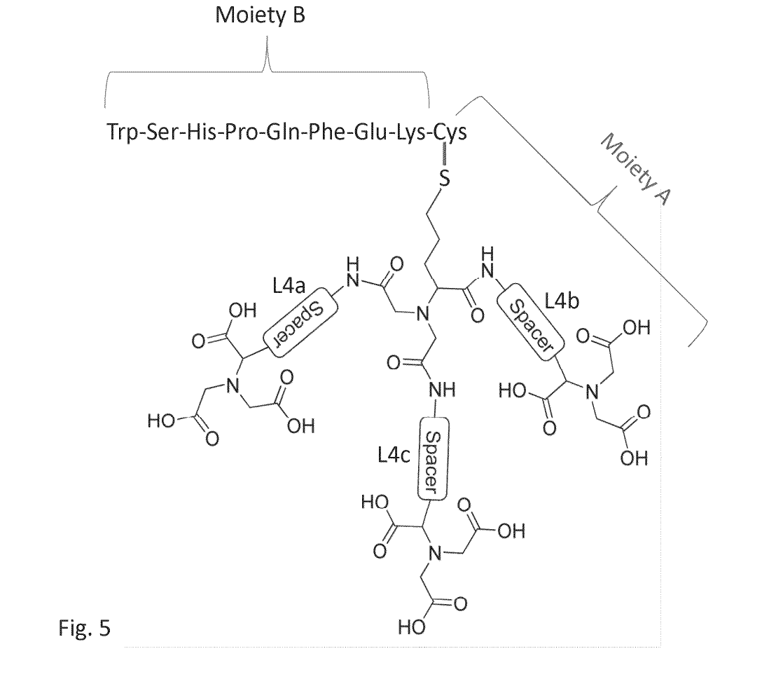

For example, the molecule -Trp-Ser-His-Pro-Gln-Phe-Glu-Lys-Cys(C₁-Cₙ₁-NTA)-Cys(C₁-Cₙ₂-NTA)-Cys(C₁-Cₙ₃-NTA)-, wherein each of (C₁-Cₙ₁-NTA), (C₁-Cₙ₂-NTA) or (C₁-Cₙ₃-NTA), in with n₁, n₂ and n₃ are independently selected from any number of 1 to 20 (including 2, 3, 4, 5, 6, 7, 8, 9, 10, 11, 12, 13, 14, 15, 16, 17, 18 and 19) is covalently linked to the sulfur (S) atom of the respective cysteine residue is a molecule falling within the scope of formula (I), with B being a streptavidin binding peptide, and the three consecutively arranged Cys residues that carry three chelating groups K, form together with the chelating groups K the moiety B. In case the three cysteine residues are directly fused/linked to the C-terminus of the streptavidin binding peptide A, no linker moiety L is present. However, as illustrated by formula (I) and formula (II) as well as FIGS. 2 to 5, if desired, virtually any linker moiety (linker) L can be present between the binding moiety A that comprises at least two chelating groups K, and the peptide based or peptidic binding moiety B. The linker L may, for example, be a peptidic linker or a straight or branched hydrocarbon based moiety. The linker L can also comprise cyclic moieties (cf. in this respect, for example, the linkage moiety L2 in FIG. 3 that has a cyclic moiety). A peptidic linker of, for example, 1 to 50, 1 to 40, 1 to 30, 1 to 20 or 1 to 10 amino acids can be arranged between the C-terminus of a moiety B such as a streptavidin binding peptide and moiety A. If the linking moiety L is a hydrocarbon-based moiety the main chain of the linker may comprise only carbon atoms but can also contain heteroatoms such as oxygen (O), nitrogen (N) or sulfur (S) atoms. The linker L may for example a C₁-C₂₀ carbon atom chain or a polyether based chain such as polyethylene based chain with —(O—CH₂—CH₂)— repeating units. In typical embodiments of hydrocarbon based linkers, the linking moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18, and 19 main chain atoms. In some embodiments of the adapter molecule as described herein, the peptidic linker comprises 1 to 6 amino acid residues that provide a side chain that is able to form a covalent bond with the at least two multidentate chelating ligands. Examples of suitable amino acid residues that provide a side chain able to form a covalent bond with a multidentate chelating ligand include, but are not limited to, cysteine, serine, threonine, tyrosine, lysine, glutamate, aspartate and combinations thereof.

In embodiments of an adapter molecule described here, it is of course also possible to fuse a Cys(C₁-Cₙ₁-NTA)-Cys(C₁-Cₙ₂-NTA)-Cys(C₁-Cₙ₃-NTA), wherein in each of (C₁-Cₙ₁-NTA), (C₁-Cₙ₂-NTA) or (C₁-Cₙ₃-NTA), n₁, n₂ and n₃ is each independently selected from 1 to 20, including each n=1, 2, 3, 4, 5, 6, 7, 8, 9, 10, 11, 12, 13, 14, 15, 16, 17, 18 and 19) directly or via a linker moiety to the N-terminus of the moiety B such as a streptavidin binding peptide or any of the other affinity peptide described here. So doing results in a molecule of formula (II), A-L-B. Such a molecule might, for example, be Cys(C₁-C₁₂-NTA)-Cys(C₁-C₁₂-NTA)-Cys(C₁-C₁₂-NTA)-Trp-Ser-His-Pro-Gln-Phe-Glu-Lys or Cys(C₁-C₁₂-NTA)-Cys(C₁-C₁₂-NTA)-Cys(C₁-C₁₂-NTA)-Ala-Ala-Ala-Trp-Ser-His-Pro-Gln-Phe-Glu-Lys, wherein the main chain carbon atoms of each of the (C₁-C₁₂) groups are optionally substituted by one or more heteroatoms selected from the group of N, O and S.

In other embodiments, the adapter molecule of the invention has the formula

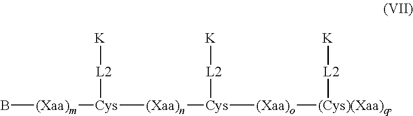

or the formula

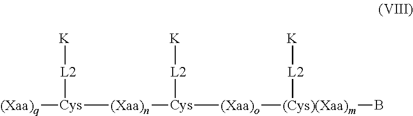

wherein Xaa is any amino acid and n, and o, have independently from each other a value of 0, 1, 2, 3 or 4, and m and q have independently from each other a value between 0 and 50 and wherein each cysteine is linked (via the sulfur (S) atom of the side chain) via an optionally present linker moiety L2 to a chelating ligand K. In the adapter molecule of formula VII or VIII the linker moiety L2, if present, may comprise 1 to 20 main chain atoms, or 1 to 15 main chain atoms, or 1 to 12 main chain atoms, or 1 to 10 main chain atoms or 1 to 2, 3, 4, 5 or 6 main chain atoms, wherein the main chain atoms are carbon atoms that are optionally replaced by one or more heteroatoms selected from the group consisting of N, O and S.

In illustrative embodiments, the adaptor molecule of the invention may have the formula (IX):

-Trp-Ser-His-Pro-Gln-Phe-Glu-Lys-Cys(C₁-Cn₁-NTA)-Cys(C₁-Cn₂-NTA)-Cys(C₁-Cn₃-NTA)-,

wherein each of (C₁-Cn₁-NTA), (C₁-Cn₂-NTA) and (C₁-Cn₃-NTA) is covalently linked to the S atom of the respective cysteine residue, or the formula (X):

-Trp-Ser-His-Pro-Gln-Phe-Glu-Lys-(Xaa)ₙ-Cys(C₁-Cₙ₁-NTA)-Cys(C₁-Cₙ₂-NTA)-Cys(C₁-Cₙ₃-NTA)-(Xaa)-Trp-Ser-His-Pro-Gln-Phe-Glu-Lys-(SEQ ID NO: 9),

wherein Xaa is any amino acid and n is an integer from 0 to 10, wherein each (C₁-Cₙ₁-NTA), (C₁-Cₙ₂-NTA) and (C₁-Cₙ₃-NTA) is covalently linked to the S atom of the respective cysteine residue. In compounds of formula (IX) and (X) each of n₁, n₂ and n₃ may have a value independently selected from 1, 2, 3, 4, 5, 6, 7, 8, 9, 10, 11, 12, 13, 14, 15, 16, 17, 18, 19, or 20, and the main chain carbon atoms of each of the C₁-Cₙ₁, C₁-Cₙ₂ or C₁-Cₙ₃ group may be optionally substituted by one or more heteroatoms selected from the group consisting of N, O and S.

As seen from the above examples it is evident that indeed a myriad of possible adapter molecule exists. It is also noted that instead of a streptavidin binding affinity peptide, any other affinity tag such as the Myc-tag (sequence: EQKLISEEDL) or the FLAG-tag (sequence: DYKDDDDK) can be used. This includes also sequential arrangements of at least two affinity modules such as “tandem” or “triple” tags such as DYKDDDDK-Gly-Gly-Gly-Ser-Cys-Cys-Cys-Gly-Gly-Gly-Ser-DYKDDDDK. Also mixtures of different affinity tags may be included in the adapter such as, e.g., WSHPQFEK-Gly-Gly-Gly-Ser-Cys(C₁-C₁₂-NTA)-Cys (C₁-C₁₂-NTA) Cys(C₁-C₁₂-NTA)-Ser-Gly-Gly-Gly-DYKDDDDK, to introduce more than one further affinity tag to the oligohistidine fused target protein.

Any chelating group K can be used in the binding moiety A as long as the chelating group K is capable of binding to a transition metal ion, thereby rendering moiety A capable of binding to an oligohistidine affinity tag. Since the moiety A of an adapter molecule as described here comprises at least two chelating groups K, these at least two groups K can be identical or different.

In some embodiments of the adapter molecule at least one or both of the at least two chelating groups K are multidentate chelating groups (ligands). In some embodiments, the moiety comprises 3, 4 or 5 such multidentate chelating groups (ligands). Examples of suitable multidentate chelating groups include, but are not limited to a polyamino carboxylic acid, a polyamine compound such as tris(2-aminoethyl) amine (TREN) and combinations of polyamino carboxylic acids and polyamine compounds.

Examples of suitable polyamino carboxylic acids that can be used as ligand K include, but are not limited, to an iminodiacetic acid (IDA), an ethylenediaminetetraacetic acid (EDTA), a nitrilotriacetic acid (NTA), a diethylene triamine pentaacetic acid (DTPA), an ethylene glycol tetraacetic acid (EGTA), 2,2′,2″-(1,4,7-triazonane-1,4,7-triyl)triacetic acid (NOTA), an 1,4,7,10-tetraazacyclododecane-1,4,7,10-tetraacetic acid (DOTA), carboxymethylated aspartic acid (CM-Asp), tris(carboxymethyl)-ethylenediamine (TED) and combinations thereof.

In some embodiments which use a nitrilotriacetic acid (NTA) based ligand K, the NTA based ligand K forms a chelating such bis-nitrilotriacetic acid (bis-NTA), tris-nitrilotriacetic acid (tris-NTA), tetrakis-nitrilotriacetic acid (tetrakis-NTA) or any combination thereof.

In some embodiments, adapter molecules (reagents) of low molecular weight are advantageous. A low molecular weight adapter should be of a molecular weight of below 20,000 Da, more preferably below 10,000 Da, even more preferably below 8,000 Da, even more preferably below 6,000 Da, even more preferably below 5,000 Da, even more preferably below 4,000 Da and most preferably below 3,000 Da. As the affinity adapter does not have to be produced by a host organism, the affinity tag does not have to be proteinaceous. In accordance with the disclosure above, it may therefore be interesting to use, for example, those affinity tag systems that use large fusion peptides as affinity tag, like, e.g., the GST and MBP system, in an inverse manner. This means that the small affinity ligands for these fusion peptides, i.e. glutathione (which is a peptide based affinity tag as defined herein) and maltose, respectively, may be equipped with the at least 2 chelating groups to generate an adapter molecule of the invention and the high molecular weight components GST and MBP, respectively, are bound to the resin for binding and thereby separating the adapter bound oligohistidine tag fusion protein.

Summarizing, the benefit of adapter reagents (adaptor molecules) of the present invention is based mainly on i) the more selective binding of soluble adapter reagents, comprising multiple but limited in number and spatially constrained transition metal ions, to the oligohistidine tag target protein thereby avoiding resins equipped at high density with transition metal ions and ii) the subsequent possibility of performing the actual chromatographic purification step on a resin that avoids the disadvantages as compared to purifying via IMAC.

It is further advantageous to couple an adapter molecule (reagent) of the invention with a label, such as, but not limited to, a fluorescent label, a chromophoric label, a spin label, a radioactive label or a magnetic label. For example, an adaptor molecule may be equipped with a fluorescent dye (label) or with a chromophore (chromophoric label) to establish a means for easily detecting or monitoring (tracking) the whereabouts of the target protein. This is, e.g., helpful during the affinity purification process addressed by the respective affinity tag entity provided within the adapter reagent of the invention. E.g. in this way the optimal amount of crude lysate to be applied to the respective affinity column, which is accomplished when the adapter reagent labeled target protein occupies 80-90% of the binding sites that are available on the column, can be easily determined during the loading process. This makes it simple to ensure that a maximal yield of the adapter reagent labeled target protein for a given volume of affinity resin and a given crude lysate is obtained within one single chromatographic step. Further, the label may be helpful for directly monitoring elution of the target protein from the column and, subsequently, also for monitoring the optional removal of the labeled adapter reagent from the target protein through, e.g., dialysis. Removal may be, e.g., facilitated by the addition of EDTA (e.g. 1 mM) to the dialysis buffer.

Equally, equipping the adapter reagent of the invention with a chromophoric and/or fluorescent label facilitates the determination of the appropriate amount of adapter reagent to be added to the crude lysate for optimal labeling of the oligohistidine tag target protein, i.e., without limitation, to label >70%, more preferably >80%, more preferably >90%, more preferably >95%, more preferably >99% of the oligohistidine tag target protein without using a significant excess of adapter reagent. This can be, e.g., achieved by a small scale titration experiment where different amounts of adapter reagent are added to equal portions of the crude lysate with the unknown content of oligohistidine tag target protein and subjecting said portions to analytical size exclusion chromatography. Conditions are selected following the sample which produces the strongest (fluorescence or chromophoric) signal at the expected molecular weight of the oligohistidine tag target protein in complex with the adapter reagent in combination with the lowest signal produced at a molecular weight expected to be generated by the adapter reagent alone.

A further advantage of an adapter reagent of the invention is provided by the possibility to adjust the amount of target protein to be purified to the capacity of a given affinity column. This can simply be achieved by adding an amount of adapter reagent of the invention known to optimally load the given affinity column to a protein mixture which may be a crude lysate containing the oligohistidine tag target protein in excess. The advantage is that it is not necessary to determine the exact content of the target protein in the protein extract because the amount that will be directed to the affinity column is determined by the known amount of adapter reagent added to the mixture. And such optimal amount of adapter reagent needs to be determined only one time for an affinity column of a given size. In this way, standardized affinity columns having a certain binding capacity can be ideally loaded with adapter reagent bound target protein in each affinity chromatography.

Yet a further advantage of an adapter reagent of the invention is provided by the possibility to further purify oligohistidine tag fusion proteins that have already been purified via IMAC without the need of further modifying the target protein through direct fusion of an affinity tag other than the oligohistidine tag. IMAC is known to be not as selective as other tag based affinity chromatography methods like, e.g., Strep-Tactin® affinity chromatography. This drawback is often foreseen and tried to be remedied in advance by fusing a further affinity tag to the other terminus of the recombinant target protein or to combine the further tag directly with the oligohistidine tag at the same end of the target protein during the cloning process using appropriate expression vectors. This approach has, however, the drawback that the recombinant target protein needs to be further modified by the further affinity tags which may result in folding and/or activity problems or in problems such as that both affinity tags negatively influence each other or are susceptible to proteolytic digestion when attached sequentially. The reversibly binding adapter reagents of the invention enable a second affinity chromatography purification step on a further affinity resin with non-specific binding properties completely other than IMAC to obtain the target protein at the desired purity without the need for further covalent modification of the target protein. For this purpose of further purification of an already IMAC purified target protein with only an oligohistidine tag attached, imidazole, that is usually used for elution of the oligohistidine tag fusion protein from the metal ion loaded resin during IMAC and that would hamper binding of the adapter reagent of the invention to the oligohistidine target protein in the IMAC eluate, has first to be removed by any known methodology, e.g., via dialysis or size exclusion chromatography or ultrafiltration. Then, a stochiometric amount or a slight excess of adapter reagent with respect to the target protein is added to the target protein solution (in, e.g., PBS pH8) and, after a short incubation step, the whole sample is applied to, e.g., a column that is filled with an appropriate amount of affinity resin, e.g. Strep-Tactin® Superflow®, that is addressed by moiety B of the used adapter reagent, e.g., a streptavidin binding peptide such as the Strep-Tag®. In case of using Strep-Tactin® affinity chromatography as second purification step (Strep-Tactin® is the trademark name for the streptavidin muteins Va144-Thr45-Ala46-Arg47 and Ile44-Gly45-Ala46-Arg47, both of which are described in U.S. Pat. No. 6,103,493), the column may be washed with PBS pH8 and the further purified adapter reagent bound oligohistidine target protein may be eluted from the column by adding 5 mM desthiobiotin in PBS pH8. Alternatively, elution may also be performed by the addition of 5 mM EDTA in PBS pH8. In this case, the adapter reagent remains on the Strep-Tactin® affinity column and the target protein is eluted free of adapter reagent. The actual procedure usually depends on the intended further use of the target protein. This subsequent use may be based on using further reagents binding to the Strep-Tag® affinity peptide (moiety B of the adapter), like, e.g., antibodies like StrepMAB-Immo, or on using further reagents binding to the oligohistidine tag directly fused to the target protein, like, e.g., antibodies like Penta-His. The procedure of obtaining the target protein free of adapter reagent may also be useful if the binding of a further adapter reagent is intended to perform a further affinity purification step on a further affinity resin.

Yet a further advantage of the reversibly binding adapter reagents of the invention is that even a multitude of sequential different affinity purification steps is possible for a target protein being fused to one affinity tag only being the oligohistidine tag. In this case, different adapter molecules having different affinity tags as moiety B have to be used in a sequential mode. This necessitates the removal of the actual adapter reagent from the oligohistidine tag fusion protein during or after each elution from the affinity resin addressed by moiety B of the actual adapter reagent prior to binding the next adapter reagent of the invention containing an affinity tag other than the affinity tag of the actual adapter reagent and other than the oligohistidine tag to the recombinant oligohistidine tag target protein. Removal of the adapter reagent may, e.g., be accomplished by dialyzing the eluate against a buffer containing EDTA using a dialysis tubing having a molecular weight cut off appropriate for separating the adapter reagent from the target protein. Prior to binding the next adapter reagent, EDTA has to be removed as well by, e.g., further dialysis against a Ni-NTA binding buffer (e.g. pH8 buffer without EDTA) that is suitable to allow binding of moiety B of said next adapter reagent to the corresponding affinity resin for the subsequent purification step.

Yet a further advantage of an adapter reagent of the invention is provided by the flexible use in further applications after purification. If, for example, an adapter reagent is used that brings a streptavidin binding peptide such as the Strep-Tag® to the oligohistidine tag fusion protein, it can be freely chosen whether the target proteins' further use—after purification—should be facilitated by using the “oligohistidine tag world of accessory reagents”, e.g., without limitation, reporter enzyme labeled antibodies directed against oligohistidines for detection purposes or trisNTA modified chips for immobilization purposes or by using the “Strep-Tag® world of accessory reagents”, e.g., without limitation, reporter enzyme labeled antibodies directed against Strep-Tag® for detection purposes (e.g. StrepMAB-Classic) or StrepMAB-Immo modified microtiter plates (available from IBA GmbH, Gottingen, Germany) for immobilization purposes. In the first case, the adapter reagent has to be removed to enable the binding of said oligohistidine tag binding reagents, in the second case, removal of the adapter reagent is not required.

Yet a further advantage of an adapter reagent of the invention is that an adapter reagent having a moiety B consisting of a solubility mediating protein like maltose binding protein (MBP) or NusA or a Small Ubiquitin-like Modifier protein (SUMO) or other solubility mediating proteins may be bound to a oligohistidine tag target protein solubilized from, e.g., inclusion bodies to enhance the yield of the functional protein during the refolding process consisting of removing the solubilizing chaotropic salt.

Yet a further advantage of an adapter reagent of the invention is that an adapter reagent having a moiety B consisting of a protein catalyzing refolding processes by catalyzing rate limiting folding steps like prolyl cis:trans isomerization or disulfide bond exchange like prolyl cis:trans isomerase or protein disulfide isomerase or other proteins known to have a chaperone effect may be bound to a oligohistidine tag target protein solubilized from, e.g., inclusion bodies to enhance the yield of the functional protein during the refolding process consisting of removing the solubilizing chaotropic salt. In this case, a longer linker between moiety B and moiety A is advisable to allow the catalytic protein of moiety B to reach the respective rate limiting folding domains of the target protein. The increase of active concentration of the chaperone in close proximity to the target protein to be refolded may enhance the refolding process considerably versus the situation where both reactants are free in solution. Also more than one chaperone may be brought to the target protein by using a moiety B consisting of combined or fused chaperones.

In accordance with the above, the invention also provides a method of equipping a fusion protein carrying an oligohistidine affinity tag with a further reversible affinity tag, the method comprising
contacting the fusion protein carrying an oligohistidine affinity tag with a bifunctional adapter molecule as described herein,
and allowing forming a complex between the adaptor molecule and the oligohistidine affinity tag.

This method may further comprise contacting the fusion protein to which the adapter molecule is bound with a solid phase comprising a binding partner for the (peptide or carbohydrate based) affinity tag of moiety B of the adapter molecule, thereby immobilizing the fusion protein on the solid phase. This method may then further comprise disrupting the reversible bond formed between the peptide or carbohydrate based affinity tag of moiety B of the adaptor molecule and the binding partner for the peptide or carbohydrate based affinity tag of moiety B. This method may also further comprise disrupting the reversible bond formed between the oligohistidine affinity tag of the target protein and moiety A of the adapter molecule. Thus, in embodiments of this method the fusion protein is purified.

The invention also provide a method of improving the solubility or folding efficiency of a protein of interest, the protein of interest carrying an oligohistidine affinity tag, wherein the method comprises
contacting the protein of interest carrying an oligohistidine affinity tag with a bifunctional adapter molecule comprising two binding moieties A and B, wherein the binding moiety A of the adapter molecule comprises at least two chelating groups K, wherein each chelating group is capable of binding to a transition metal ion, thereby rendering moiety A capable of binding to an oligohistidine affinity tag, and the binding moiety B is an peptide based affinity tag other than an oligohistidine tag,
and allowing forming a complex between the adaptor molecule and the oligohistidine affinity tag.

In embodiments of this method the binding moiety B comprises or is a solubility mediating protein. Examples of suitable solubility mediating protein include maltose binding protein (MBP), NusA or a Small Ubiquitin-like Modifier protein (SUMO). In this method the binding moiety B may be a protein that catalyzes a refolding process or a protein having a chaperon effect. The protein that catalyses a refolding process may, for example, be a prolyl cis:trans isomerase or protein disulfide isomerase.

The invention will be further illustrated by the non-limiting Examples.

### Examples

### Example A

### Exemplary Synthesis of an Exemplary Adapter Reagent (Oligohistidine-> Strep-Tag®II) of the Invention

Coupling of a Fluorescence Labeled Peptide Comprising the Strep-Tag®II Affinity Peptide with Maleimido-C3-NTA

A chelator/peptide adapter reagent can be simply synthesized by reacting cysteine residues with maleimide activated nitrilotriacetic acid compounds using one of the two following methods.

### Method 1

In a first step, the following labeled peptide, FITC-Ahx-SAWSHPQFEKCCC (MW=2028.30 g/mol), is synthesized. The fluorescein isothiocyanate (FITC) fluorescence label is optional and the peptide might also be used without FITC label, i.e., without limitation, in the version SAWSHPQFEKCCC. The peptide comprises Strep-tagII (WSHPQFEK) providing affinity for the streptavidin mutein called Strep-Tactin® (that is commercially available from IBA GmbH, Gottingen, Germany and is also described in U.S. Pat. No. 6,103,493). The peptides (with or without fluorescence label) are standard peptide products and can be synthesized by standard methods in peptide chemistry. The Strep-Tag® moiety (affinity tag) is extended with at least two cysteine residues, whereby said cysteine residues need not necessarily be appended in a direct consecutive manner. Also building blocks other than cysteines may be added as long as they provide at least two selectively addressable chemical groups (that are not present in the affinity tag or label moieties) for site specific reaction with, e.g., correspondingly activated NTA.

In a second step, maleimide activated NTA (maleimido-C3-NTA) was coupled to the cysteine derived thiol groups which is preferably performed in solution in the presence of TCEP (in PBS) to keep the cysteines in a reduced state. After a short incubation step with TCEP, a molar excess of maleimido-C3-NTA (Dojindo Molecular Technologies, Inc.) was added and the mixture was reacted overnight at RT. The following day, excess maleimido-C3-NTA was removed by binding the peptide to immobilized Strep-Tactin® and washing. Through addition of Ni²⁺-containing buffer to the immobilized NTA labeled peptide, NTA groups were loaded with Ni²⁺. Excess of Ni²⁺ was removed by washing and the metal ion loaded peptide was eluted from the column by the addition of desthiobiotin. Adapter reagent identity is confirmed by mass spec and is then ready for use. Desthiobiotin can be optionally removed by gel filtration or dialysis. Alternatively, to avoid the addition of a competing compound in cases where said competing compound hampers further use, elution of the adapter reagent from the Strep-Tactin® column can be performed with a pH 5 buffer without containing desthiobiotin which can be more readily changed to conditions appropriate for further use, simply by adding an excess of a buffer substance providing the appropriate pH and ionic strength.

### Protocol:

1. 1 mg of peptide was dissolved in 1 ml PBS, 2 mM TCEP, and pH was confirmed to be pH 7.5. The solution was incubated at RT for 1-2 hours to reduce the cysteines.
2. The solution was used to dissolve 10 mg of maleimido-C3-NTA (Dojindo) and incubated overnight at RT in the dark.
3. The following morning, the solution was applied to a Strep-Tactin Superflow high capacity gravity flow column (CV=5 ml) pre-equilibrated with PBS.
4. The column was washed with 2×5 ml PBS to remove excess maleimido-C3-NTA.
5. The column was washed with 2×5 ml PBS containing 10 mM NiCl₂ to load the NTA groups.
6. The column was washed with 2×5 ml PBS.
7. The adapter reagent with NTA groups loaded with Ni²⁺ metal ions was eluted by the addition of PBS containing 2.5 mM desthiobiotin or by 50 mM acetate buffer pH 4.8.
8. Optionally desthiobiotin was removed or the buffer was changed by dialysis against a buffer appropriate for further use.
9. The extent of NTA coupling and load with Ni²⁺ ions and labeling was characterized by mass spectrometry and the adapter reagent was quantified (e.g. through the FITC label).

### Method 2

In a first step, the following labeled peptide, 5(6)-Carboxyfluorescein-Ahx-SAWSHPQFEKCCC—NH2 (MW=1996.22 g/mol), was synthesized with C-terminal amide and lyophilized in 1 mg aliquots OPT Peptide Technologies GmbH, Germany). The fluorescein fluorescence label is optional and the peptide might also be used without fluorescein label, i.e., without limitation, in the version SAWSHPQFEKCCC. The peptide comprises Strep-tagII (WSHPQFEK) providing affinity for the streptavidin mutein called Strep-Tactin® (that is commercially available from IBA GmbH, Gottingen, Germany and is also described in U.S. Pat. No. 6,103,493). The peptides (with or without fluorescence label) are standard peptide products and can be synthesized by standard methods in peptide chemistry. The Strep-Tag® moiety (affinity tag) is extended with at least two cysteine residues, whereby said cysteine residues need not necessarily be appended in a direct consecutive manner. Also building blocks other than cysteines may be added as long as they provide at least two selectively addressable chemical groups (that are not present in the affinity tag or label moieties) for site specific reaction with, e.g., correspondingly activated NTA.

In a second step, maleimide activated NTA (maleimido-C3-NTA, Dojindo Molecular Technologies, Inc.) was coupled to the cysteine derived thiol groups which is preferably performed in solution in the presence of TCEP (in phosphate buffer) to keep the cysteines in a reduced state. After a short incubation step with TCEP, a molar excess of maleimido-C3-NTA was added and the mixture was reacted overnight at RT. The following day, excess maleimido-C3-NTA was removed by binding the peptide to immobilized Strep-Tactin® and washing. Through addition of Ni²⁺-containing buffer to the immobilized NTA labeled peptide, NTA groups were loaded with Ni²⁺. Excess of Ni²⁺ was removed by washing and the metal ion loaded peptide was eluted from the column by the addition of desthiobiotin. Adapter reagent identity can be confirmed by mass spec and is then ready for use to label oligohistidine tag fusion proteins. If purification of the oligohistidine tag fusion protein is intended by binding the 5(6)-carboxyfluorescein-Ahx-SAWSHPQFEKCCC—NH2 peptide comprising NTA groups at the cysteine side chains and subsequent affinity chromatography on immobilized Strep-Tactin®, desthiobiotin is removed by ultrafiltration, gel filtration or dialysis because desthiobiotin would compete and thereby hamper in this case the binding of the adapter reagent labeled oligohistidine tag fusion protein to immobilized Strep-Tactin®. Alternatively, to avoid the addition of a competing compound such as desthiobiotin in cases where said competing compound hampers further use, elution of the adapter reagent from the Strep-Tactin® column can be performed also with a pH 5 buffer (based on, e.g., acetate) without containing desthiobiotin which can be more readily changed to conditions appropriate for further use like purification, simply by adding an excess of a buffer substance providing the appropriate pH and ionic strength. The extent of NTA coupling and load with Ni²⁺ ions and labeling may be characterized by, e.g., mass spectrometry. Quantification of the adapter reagent may be performed by, e.g., absorption spectroscopy by using, e.g. tryptophan and/or tyrosine absorbance at 280 nm or by using fluorescein absorbance at 494 nm.

### Protocol:

1. 1 mg of lyophilized peptide (5(6)-Carboxyfluorescein-Ahx-SAWSHPQFEKCCC—NH2) was dissolved in 1 ml phosphate/TCEP buffer (200 mM sodium phosphate, 115 mM NaCl, 2 mM TCEP, pH 7.4). The solution was incubated at RT for 1-2 hours to reduce the cysteines.
2. The solution was then used to dissolve 10 mg of maleimido-C3-NTA (Dojindo) and incubated overnight at RT in the dark.
3. The following morning, the solution was applied to a Strep-Tactin Superflow high capacity gravity flow column (column volume (CV)=4 ml, IBA GmbH) pre-equilibrated with PBS pH8 (=Dulbecco's PBS from PAA Laboratories GmbH, Austria, adjusted to pH 8 with NaOH).
4. The column bound adapter reagent was washed with 2×5 ml PBS pH 8 and 1× with TBS (100 mM Tris-Cl pH 8, 115 mM NaCl) to remove excess maleimido-C3-NTA.
5. The column was washed with 2×5 ml TBS containing 10 mM NiCl₂ to load the NTA groups.
6. The column was washed with 2×5 ml TBS.
7. The adapter reagent with NTA groups loaded with Ni²⁺ metal ions was eluted by the addition of TBS containing 5 mM desthiobiotin. The eluate containing the adapter reagent was easily visually detectable through the fluorescein label and amounted to 4 ml.
8. Desthiobiotin was removed through several concentration/dilution steps via ultrafiltration. For dilution, PBS pH 8 was used. Concentration via ultrafiltration was achieved by using a Vivaspin 15R device (molecular weight cut off=2000 Da, Sartorius Stedim Biotech GmbH) and centrifugation at 3000 g at 4° C. In a first step, desthiobiotin was diluted to less than 100 nM by this concentration/dilution procedure. Then, the Vivaspin device containing 1 ml of the concentrated adapter reagent was diluted with 14 ml PBS pH8 containing 50 μM NiCl₂ to load NTA groups which may have potentially lost its Ni²⁺ ion during the prolonged concentration/dilution procedure. Then, a further 1:200 dilution was achieved with further concentration/dilution steps using PBS pH8 to dilute excess NiCl₂ to below 0.25 μM. Finally, the adapter reagent was concentrated to 1.1 ml and dissolved in PBS pH8 containing residual NiCl₂ (<0.25 μM) and residual desthiobiotin (<1 nM).
9. The finally obtained adapter reagent was quantified through measuring absorbance at 494 nm which equalled to 12.85. Using an extinction coefficient of 70000 M⁻¹ cm⁻¹ (published by Thermo Scientific for 5(6) carboxyfluoresceine succinimidyl ester), a concentration of 184 μM was deduced for the adapter reagent. This corresponds to a yield of 202 nmol adapter reagent (corresponding to a yield of 40% as the procedure was started with 500 nmol peptide). The extent of NTA coupling and load with Ni²⁺ ions and labeling was not further characterized. Successful adapter reagent synthesis was indirectly demonstrated by successful use of the adapter reagent produced by this procedure for purification of GFP with C-terminal oligohistidine tag (6× His-tag) from a crude bacterial extract via Strep-Tactin® affinity chromatography as described below.

An alternative way to the above-described methods to generate adapter molecules of the invention may be easily achieved with the following reagent published by Lata et al., 2005, supra, scheme 1 (B), 19:

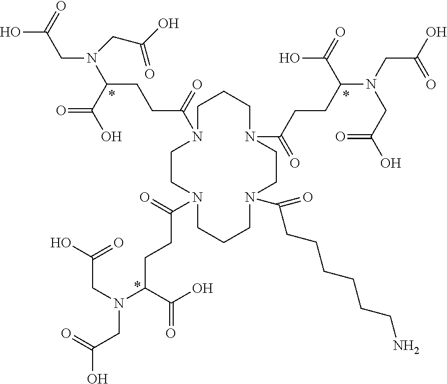 [SMILES: NCCCCCCC(=O)N1CCCN(C(=O)CCC(C(=O)O)N(CC(=O)O)CC(=O)O)CCN(C(=O)CCC(C(=O)O)N(CC(=O)O)CC(=O)O)CCCN(C(=O)CCC(C(=O)O)N(CC(=O)O)CC(=O)O)CC1]

This tris-NTA compound is a binding moiety A that comprises at least two chelating groups K (here, the three chelating groups being NTA). Each chelating group K is capable of binding to a transition metal ion, thereby rendering moiety A capable of binding to an oligohistidine affinity tag. This binding moiety A also possesses, coupled via a linker, a selectively addressable single primary amino group by means of which it can be easily coupled to a binding moiety B being an affinity tag other than an oligohistidine tag. This binding moiety B (affinity tag other than oligohistidine tag) needs an N-hydroxysuccinimide activated carboxyl group or an otherwise activated group that is able to react with primary amines. The coupling/activation chemistry for formation of the amide bond in the resulting adapter molecule (by reacting the amine group with an activated carboxyl group of moiety B) is known to the person skilled in the art of peptide chemistry (e.g. is routinely used for coupling of epitope tags or glutathione or streptavidin binding peptides), skilled in the art of protein chemistry (e.g. for coupling with protein A and derivatives and/or GST and/or MBP) and/or skilled in the art of sugar chemistry (e.g. to be used for maltose). Beyond moiety A above, described by Lata et al., 2005, supra, many other building blocks providing a moiety A comprising a reactive (addressable) chemical group and at least two chelating groups K may be synthesized by the chemist skilled in the art and coupled to an accordingly activated moiety B. Of course, coupling can also be performed in the inverse manner, i.e., equipping moiety B with a selectively addressable group and activating moiety A accordingly.

### Example B

### Use of the Adapter Reagent for Purification of an Oligohistidine Tag Fusion Protein Via Strep-Tactin® Affinity Chromatography and Improved Performance Versus Direct Purification on a Ni-NTA Resin

### Example B.1

### Glyceraldehyde 3-phosphate dehydrogenase (GAPDH)

The gene for the 37 kDa protein GAPDH from B. subtilis was cloned in the bacterial expression vector pASG-IBA33 for C-terminal fusion of a hexahistidine tag and expressed in E. coli by using the tetracycline promoter according to the following standard protocol:

### Material

Ampicillin stock solution: 100 mg/ml in H₂O, sterile filtered.
Anhydrotetracycline stock solution: 2 mg/ml in dimethylformamide (DMF).
LB medium: 10 g/l trypton, 5 g/l yeast extract, 5 g/l NaCl; sterile (autoclaved)
5× SDS-PAGE sample buffer: 0.250 M Tris.Cl, pH 8.0; 25% glycerol; 7.5% SDS, 0.25 mg/ml bromophenolblue; 12.5% v/v mercaptoethanol
E. coli strain: BL21
Lysozyme
CV=column bed volume
Strep-Tactin Superflow (5 mg/ml) for gravity flow purification
Buffer W (washing buffer): 100 mM Tris.Cl, 150 mM NaCl, pH 8
Buffer E (elution buffer): 100 mM Tris.Cl, 150 mM NaCl, 2.5 mM desthiobiotin, pH 8.
The composition of the lysis, wash and elution buffers can be modified to suit the particular application, e.g. by adding 0.1% Tween, 5-10 mM beta-mercaptoethanol, or 1 mM PMSF, or increasing NaCl concentrations. The pH should not be lower than 7.5, though.

### Protocol:

1. Pre-culture: 2 ml of LB medium containing 100 μg/ml ampicillin are inoculated with a fresh colony harboring the pASG-IBA33(GAPDH) expression plasmid and shaken overnight (200 rpm) at 37° C.

The colony should not be older than 1 week.
2. Culture for expression: 100 ml of LB medium containing 100 μg/ml ampicillin are inoculated with the pre-culture and shaken at 37° C.
3. Optical density of the culture is monitored at 550 nm (OD₅₅₀).
4. When OD₅₅₀ equals 0.5-0.6, 10 μl of the anhydrotetracycline stock solution were added to induce GAPDH expression.
5. Expression is continued for 3 hours at 200 rpm.
6. Cells are harvested by centrifugation at 4500×g for 12 minutes (4° C.).
7. The cells are resuspended with 10 ml Buffer W.
8. Lysozyme is added to 1 mg/ml and the mixture is incubated on ice for 30 minutes.
9. The cell suspension is sonicated on ice. Six 10 second bursts at 200-300 W are used with a 10 second cooling period between each burst. A sonicator equipped with a microtip has been used.
10. (Optional) If the lysate was very viscous, RNase A (10 μg/ml) and DNase I (5 μg/ml) were added and the suspension was incubated on ice for 10-15 min.
11. The lysate (approximately 10 ml) was cleared by centrifugation at 30,000×g for 15 minutes at 4° C. to pellet the cellular debris.
12. The Strep-Tactin Superflow gravity flow column (CV=1 ml) was equilibrated by the addition of 2 CVs Buffer W.
13. 250 nmol of the synthesized exemplary adapter reagent loaded with Ni²⁺ (oligohistidine-> Strep-tagII) from above was added to the supernatant of step 11. Optionally, imidazole may be added to reduce background binding of the adapter reagent. To simulate the result after lower expression rates, the resulting mixture was diluted 1:10 and 1:100 with a cleared lysate prepared in the same manner as described above but starting with an E. coli colony harboring an empty expression vector.
14. 10 ml of the cell extract/adapter reagent mixture from the previous step were added to the Strep-Tactin Superflow gravity flow column and the flow through was collected.
15. The column was then washed 5 times with 1 CV Buffer W and the eluate was collected in fractions of 1 CV.
16. After washing, 6 times 0.5 CVs Buffer E were added and the eluate was collected in 0.5 CV fractions.
17. The fractions were analyzed by SDS-PAGE.

The adapter reagent could be optionally removed by dialyzing the target protein (GAPDH) containing fractions against Buffer W containing 1 mM EDTA.

### Example B.2

### Green Fluorescent Protein

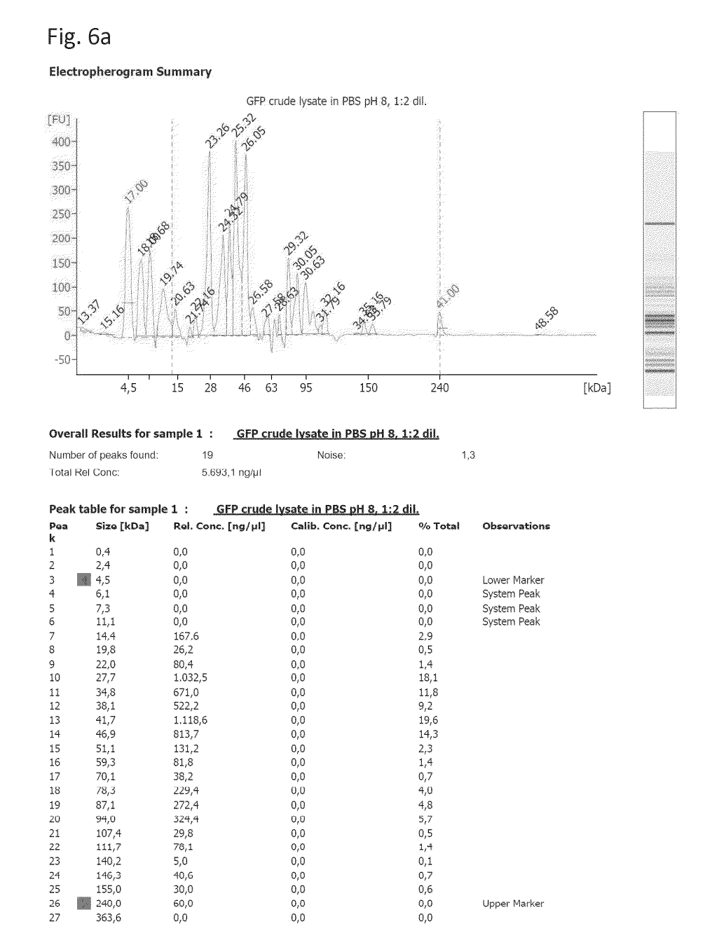

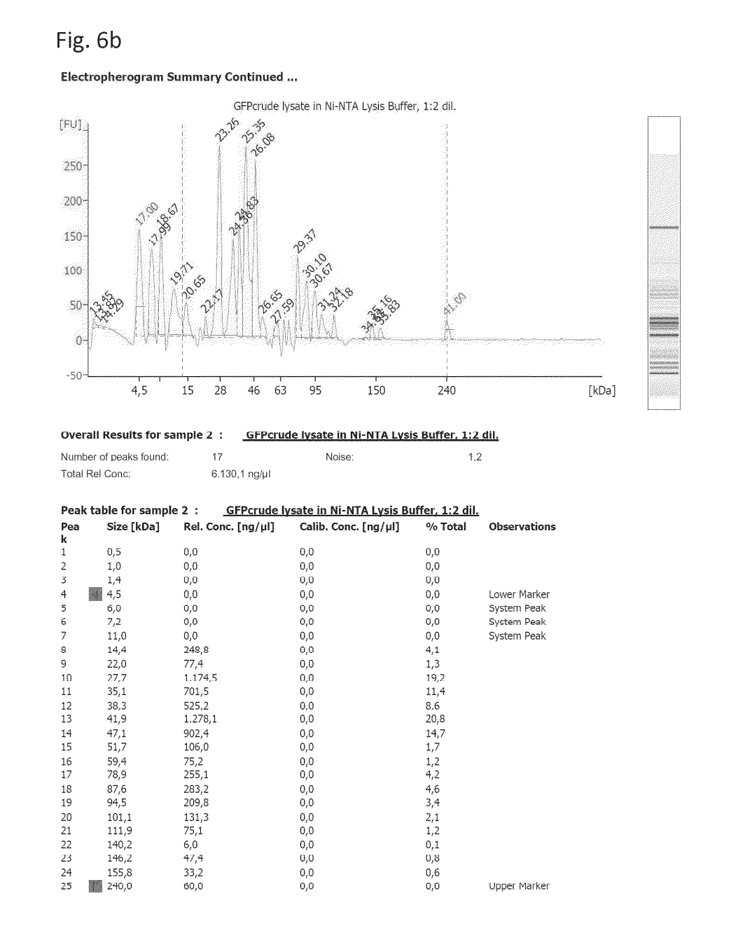

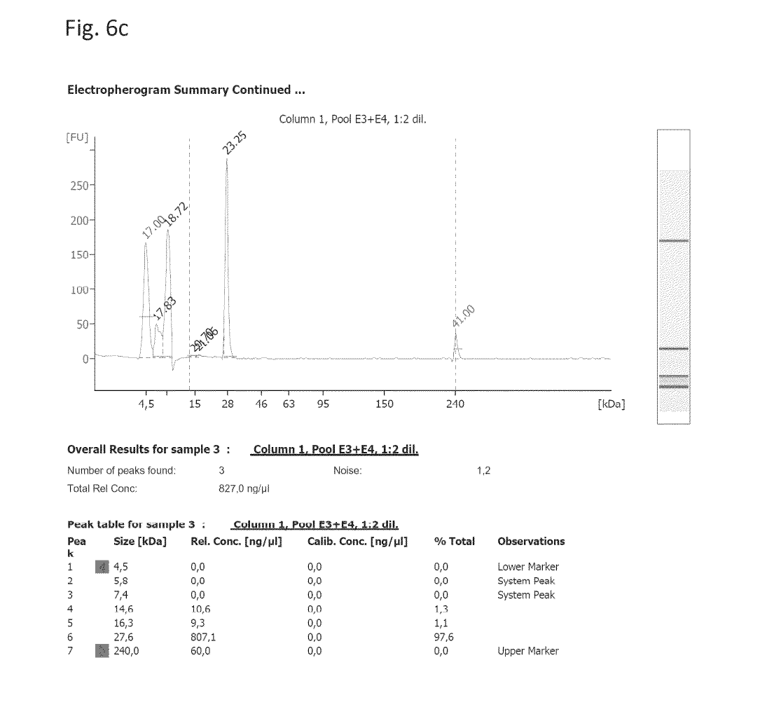

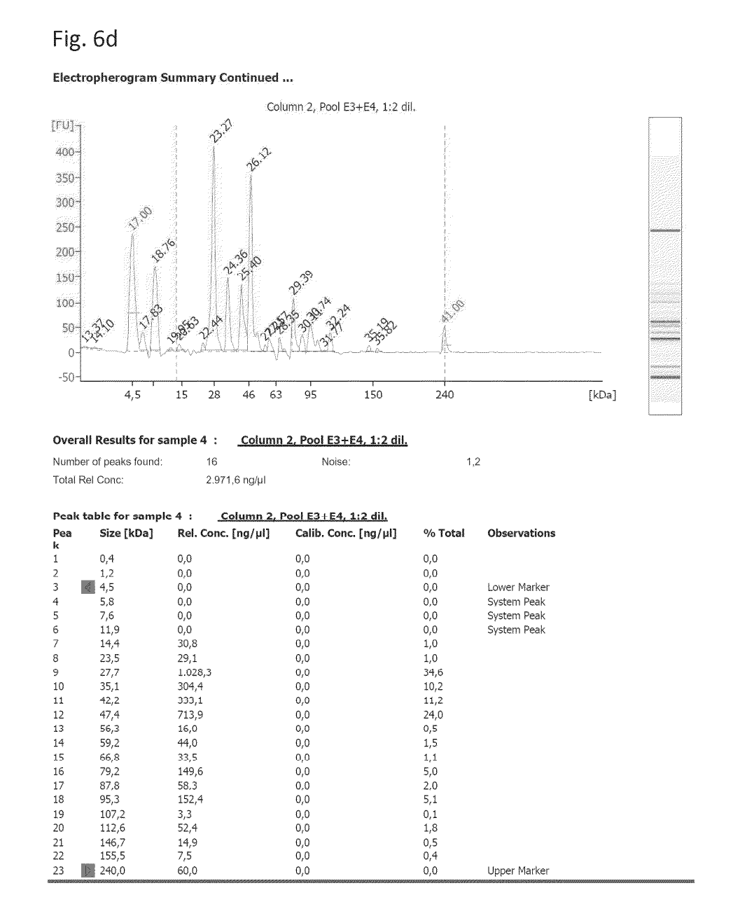

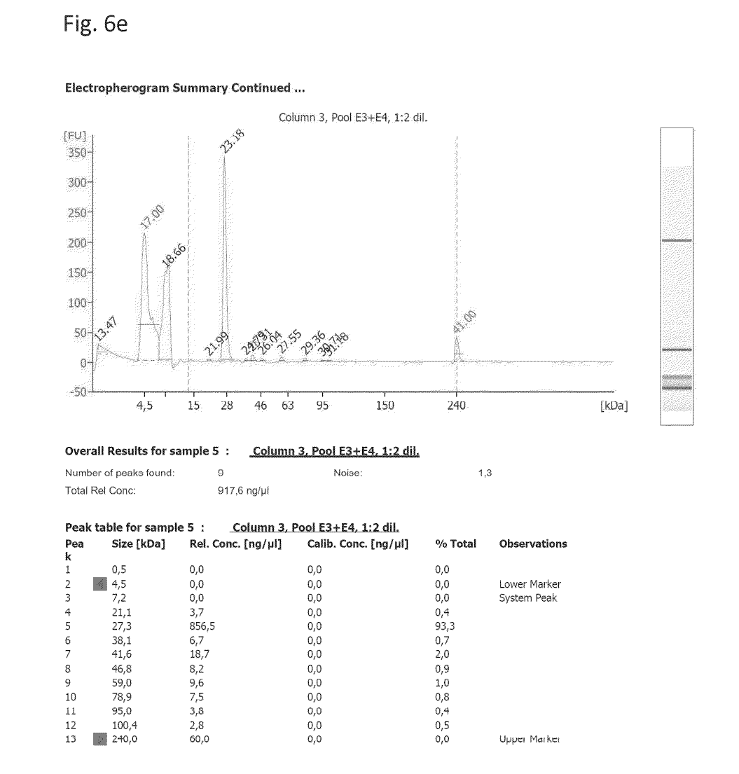

The gene for the 27 kDa protein (GFP) from A. victoria was cloned using the StarGate® cloning system (IBA GmbH) into the bacterial expression vector pPSG-IBA33. This vector thereby encodes a gene for the expression of a GFP with C-terminally fused hexahistidine tag of 27980 Da as deduced from the theoretic amino acid sequence. The recombinant gene was expressed in E. coli (BL21(DE3)) via the vector encoded T7 promoter according to a standard protocol described below. Then, the cell lysate was prepared with Ni-NTA Lysis Buffer (FIG. 6b). A fraction of the cell lysate was dialyzed (1:100 dilution) against PBS pH8 (FIG. 6a). Both lysates (in Ni-NTA Lysis Buffer (cf. FIG. 6a) and in PBS pH8 (cf. FIG. 6b)) contained approximately 30 μM GFP with C-terminally fused oligohistidine tag. Three samples were then prepared:
sample 1: 333 μl of PBS pH8 lysate were added to 66 μl of the Strep-Tag® containing adapter reagent (184 μM in PBS pH8), the preparation of which is described above, so that the final adapter reagent concentration (30 μM) was in slight excess (×1.2) over the target protein concentration (25 μM, 399 μl).
sample 2: 333 μl of PBS pH8 lysate containing 30 μM GFP-6×His.
sample 3: 333 μl of lysate in Ni-NTA Lysis Buffer containing 30 μM GFP-6×His.
   Sample 1 was applied to a 200 μl Strep-Tactin high capacity column (IBA GmbH) and each of samples 2 and 3 were subjected to IMAC on a 200 μl Ni-NTA Superflow (Qiagen GmbH) column of the same dimensions. IMAC of sample 2 was performed under physiological conditions using PBS pH8, as for Strep-Tactin chromatography, while IMAC of sample 3 was performed under non-physiological conditions in an imidazole containing high salt phosphate buffer providing optimal selectivity for IMAC.

### Material

Ampicillin stock solution: 100 mg/ml in H₂O, sterile filtered.
IPTG stock solution: 1M in H₂O, sterile filtered.
LB medium: 10 g/l trypton, 5 g/l yeast extract, 5 g/l NaCl; sterile (autoclaved)
E. coli strain: BL21(DE3)
CV=column bed volume
Strep-Tactin Superflow high capacity column for gravity flow purification (0.2 ml CV, IBA GmbH)
Ni-NTA Superflow (Qiagen GmbH) column for gravity flow purification (0.2 ml CV)
PBS pH8: Dulbecco's PBS from PAA Laboratories adjusted to pH 8 using NaOH.
Strep-Tactin Elution Buffer: PBS pH8 containing 5 mM desthiobiotin, adjusted to pH 8 with NaOH.
Ni-NTA Elution Buffer 1: PBS pH8 containing 250 mM imidazole, adjusted to pH 8 with HCl.
Ni-NTA Lysis Buffer: 50 mM NaH₂PO₄, 300 mM NaCl, 10 mM imidazole, pH 8
Ni-NTA Wash Buffer: 50 mM NaH₂PO₄, 300 mM NaCl, 20 mM imidazole, pH 8
Ni-NTA Elution Buffer 2: 50 mM NaH₂PO₄, 300 mM NaCl, 250 mM imidazole, pH 8

### Protocol:

### Expression and Lysate Preparation

1. Pre-culture: 4 ml of LB medium containing 100 μg/ml ampicillin were inoculated with a fresh colony BL21(DE3) harboring the pASG-IBA33(GFP) expression plasmid and shaken overnight (200 rpm) at 37° C. The colony should not be older than 1 week.
2. Culture for expression: 200 ml of LB medium containing 100 μg/ml ampicillin were inoculated with the pre-culture and shaken at 37° C.
3. Optical density of the culture was monitored at 550 nm (OD₅₅₀).
4. When OD₅₅₀ equaled to 0.5, 100 μl of IPTG stock solution were added to induce GFP-6×His expression.
5. Expression was continued for 48 hours at 30° C. and 200 rpm.
6. Cells were harvested by centrifugation at 4500 g for 12 minutes (4° C.).
7. The cells were resuspended with 2 ml Ni-NTA Lysis Buffer.
8. The cell suspension was sonicated on ice. Six 10 second bursts at 200-300 W are used with a 10 second cooling period between each burst. A sonicator equipped with a microtip had been used.
9. (Optional) If the lysate was very viscous, RNase A (10 μg/ml) and DNase I (5 μg/ml) were added and the suspension was incubated on ice for 10-15 min.
10. The lysate (approximately 2 ml) was cleared by centrifugation at 30,000 g for 15 minutes at 4° C. to pellet the cellular debris.
11. The supernatant containing the GFP-6×His target protein was divided in 2 parts of 1 ml. One part was dialyzed (1:100 dilution) against PBS pH8 at 4° C.
12. 3 samples were prepared:
   sample 1: 333 μl of PBS pH8 lysate were added to 66 μl of the Strep-Tag® containing adapter reagent (184 μM in PBS pH8), the preparation of which is described above, so that the final adapter reagent (approximately 30 μM in 399 μl) was in slight excess (×1.2) over the GFP-6×His target protein concentration (approximately 25 μM in 399 μl).
   sample 2: 333 μl of PBS pH8 lysate containing approximately 30 μM GFP-6×His.
   sample 3: 333 μl of Ni-NTA Lysis Buffer lysate containing approximately 30 μM GFP-6×His.

### Affinity Purification

1. Sample 1 was applied to a Strep-Tactin Superflow high capacity column for gravity flow purification (CV=0.2 ml, IBA GmbH) pre-equilibrated with PBS pH8. Samples 2 and 3 were each applied to a Ni-NTA Superflow (Qiagen GmbH) column for gravity flow purification (CV=0.2 ml)—of identical dimension as compared to the Strep-Tactin Superflow high capacity column—which were pre-equilibrated with PBS pH8 and Ni-NTA Lysis Buffer, respectively.
2. After the samples had entered the column, column 1 and 2 were washed 5 times with 0.2 ml PBS pH8 while column 3 was washed 5 times with 0.2 ml Ni-NTA Wash Buffer. The eluate at each step was collected in a separate vessel (yielding washing fractions W1-W5, each having a volume of approximately 0.2 ml).
3. Bound protein was then eluted by adding 5 times 0.1 ml elution buffer and the eluate was collected at each step in a separate vessel (yielding elution fractions E1-E5, each having a volume of approximately 0.1 ml). Column 1 was treated with Strep-Tactin® Elution Buffer, column 2 with Ni-NTA Elution Buffer 1 and column 3 with Ni-NTA Elution Buffer 2. FIG. 6c shows the result of an Agilent 2100 Bioanalyzer analysis of pooled elution fractions E3 and E4 of column 1, FIG. 6d shows the result of an Agilent 2100 Bioanalyzer analysis of pooled elution fractions E3 and E4 of column 2, and FIG. 6e shows the result of an Agilent 2100 Bioanalyzer analysis of pooled elution fractions E3 and E4 of column 3.

The adapter reagent could be optionally removed by, e.g., dialyzing the target protein (GFP-6×His) containing eluate against PBS containing 1 mM EDTA using a dialysis tubing with a molecular weight cut off of 16000 Da.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of equipping a fusion protein carrying an oligohistidine affinity tag with a further reversible affinity tag, the method comprising
contacting the fusion protein carrying an oligohistidine affinity tag with a bifunctional adapter molecule comprising two binding moieties A and B, the adapter molecule being capable of reversibly equipping a fusion protein carrying an oligohistidine affinity tag with a further affinity tag, wherein
the binding moiety A comprises at least two chelating groups K, wherein each chelating group is capable of binding to a transition metal ion, thereby rendering moiety A capable of binding to an oligohistidine affinity tag, and
the binding moiety B is an affinity tag other than an oligohistidine,
and allowing forming a complex between the adaptor molecule and the oligohistidine affinity tag.

2. The method of claim 1, further comprising contacting the fusion protein to which the adapter molecule is bound with a solid phase comprising a binding partner for the (peptide or carbohydrate based) affinity tag of moiety B of the adapter molecule, thereby immobilizing the fusion protein on the solid phase.

3. The method of claim 2, further comprising disrupting the reversible bond formed between the peptide or carbohydrate based affinity tag of moiety B of the adaptor molecule and the binding partner for the peptide or carbohydrate based affinity tag of moiety B, and optionally disrupting the reversible bond formed between the oligohistidine affinity tag of the target protein and moiety A of the adapter molecule.

4. The method of claim 2, further comprising disrupting the reversible bond formed between the oligohistidine affinity tag of the target protein and moiety A of the adapter molecule, and optionally purifying the fusion protein.

5. The method of claim 3, wherein the fusion protein is purified.

6. The method of claim 1, wherein the binding moiety B of the bifunctional adapter molecule is peptide based or carbohydrate based.

7. The method of claim 1, wherein the at least two chelating groups K of binding moiety A of the bifunctional adapter molecule are attached to different locations within the peptide based affinity tag such that the at least two chelating groups are capable of binding to the same oligohistidine affinity tag.

8. The method of claim 1, wherein the bifunctional adapter molecule has a general formula selected from the formulae
B-L-A (I),
A-L-B (II),
wherein L is an optionally present linker moiety.

9. The method of claim 1, wherein the binding moiety A of the bifunctional adapter molecule is fused, optionally via the linker moiety L, to the N-terminus or the C-terminus of the binding moiety B (peptide based affinity tag).

10. The method of claim 1, wherein the (peptide based) affinity tag of the bifunctional adapter molecule is selected from the group consisting of a streptavidin binding peptide, protein A, protein G, protein L, maltose binding protein, glutathione-S-transferase, maltose, glutathione, a calmodulin binding peptide, a chitin binding domain, a cellulose binding domain, the S-tag (sequence: KETAAAKFERQHMDS, SEQ ID NO: 18) and an epitope tag, wherein the epitope tag is preferably selected from the group consisting of the Myc-tag (sequence: EQKLISEEDL, SEQ ID NO: 11), the HA-tag (sequence: YPYDVPDYA, SEQ NO: 12), the VSV-G-tag (sequence: YTDIEMNRLGK, SEQ ID NO: 13), the HSV-tag (sequence: QPELAPEDPED, SEQ ID NO: 14), the V5-tag (sequence: GKPIPNPLLGLDST, SEQ ID NO: 15), and the FLAG-tag (sequence: DYKDDDDK, SEQ ID NO: 16).

11. The method of claim 10, wherein the streptavidin binding peptide comprises or consists of one of the following sequences:
a) -Trp-Xaa-His-Pro-Gln-Phe-Yaa-Zaa- (SEQ ID NO: 1), wherein Xaa is any amino acid and Yaa and Zaa are both Gly or Yaa is Glu and Zaa is Lys or Arg (formula III),
b) -Trp-Arg-His-Pro-Gln-Phe-Gly-Gly- (formula IV, SEQ ID NO: 2),
c) -Trp-Ser-His-Pro-Gln-Phe-Glu-Lys- (formula V, SEQ ID NO: 3),
d) a sequential arrangement of at least two streptavidin binding peptides, wherein each peptide binds streptavidin, wherein the distance between two peptides is at least 0 and not greater than 50 amino acids and wherein each of the at least two peptides comprises the amino acid sequence -His-Pro-Baa- in which Baa is selected from the group consisting of glutamine, asparagine and methionine (formula VI),
e) a sequential arrangement as recited in d), wherein one of the at least two peptides comprises the sequence -His-Pro-Gln-,
f) a sequential arrangement as recited in d), wherein one of the peptides comprises an amino acid sequence -His-Pro-Gln-Phe- (SEQ ID NO: 4),
g) a sequential arrangement as recited in d) wherein at least one peptide includes at least the amino sequence -Oaa-Xaa-His-Pro-Gln-Phe-Yaa-Zaa- (SEQ ID NO: 5), where Oaa is Trp, Lys or Arg, Xaa is any amino acid and where either Yaa and Zaa are both Gly or Yaa is Glu and Zaa is Lys or Arg,
h) a sequential arrangement as recited in d) wherein at least one peptide includes at least the amino acid sequence -Trp-Xaa-His-Pro-Gln-Phe-Yaa-Zaa- (SEQ ID NO: 6) where Xaa is any amino acid and where either Yaa and Zaa are both Gly or Yaa is Glu and Zaa is Lys or Arg,
i) a sequential arrangement as recited in d) wherein at least one peptide includes at least the amino acid sequence -Trp-Ser-His-Pro-Gln-Phe-Glu-Lys- (SEQ ID NO: 7),
j) the amino acid sequence -Trp-Ser-His-Pro-Gln-Phe-Glu-Lys-(Xaa)n-Trp-Ser-His-Pro-Gln-Phe-Glu-Lys- (SEQ ID NO: 8) wherein Xaa is any amino acid and n is an integer from 0 to 12,
k) -Trp-Ser-His-Pro-Gln-Phe-Glu-Lys-(Xaa)n-Cys-Cys-Cys-(Xaa)n-Trp-Ser-His-Pro-Gln-Phe-Glu-Lys-, (SEQ ID NO: 9) wherein Xaa is any amino acid and n is an integer from 0 to 10,
l) the amino acid sequence -Trp-Ser-His-Pro-Gln-Phe-Glu-Lys-(GlyGlyGlySer)n-Trp-Ser-His-Pro-Gln-Phe-Glu-Lys- (SEQ ID NO: 10), where n is either 2 or 3, or
m) the amino acid sequence: MDEKTTGWRGGHVVEGLAGELEQLRARLEHHPQGQREP (SEQ ID NO: 19).

12. The method of claim 2, wherein the linker moiety L of the bifunctional adapter molecule is a peptidic linker or a straight or branched hydrocarbon based linker.

13. The method of claim 12, wherein the peptidic or hydrocarbon based linker comprises 1 to 20 amino acids.

14. The method of claim 13, wherein the peptidic linker comprises 1 to 6 amino acid residues that provide a side chain that is able to form a covalent bond with the at least two multidentate chelating ligands.

15. The method of claim 1, wherein each of the at least two chelating groups (ligands) K of the bifunctional adapter molecule is a multidentate chelating ligand.

16. The method of claim 15, wherein each of at least two multidentate chelating groups (ligands) is selected from the group consisting of a polyamino carboxylic acid, a polyamine compound and combinations thereof.

17. The method of claim 15, wherein the polyamino carboxylic acid is selected from the group consisting of an iminodiacetic acid (IDA), an ethylenediaminetetraacetic acid (EDTA), a nitrilotriacetic acid (NTA), a diethylene triamine pentaacetic acid (DTPA), an ethylene glycol tetraacetic acid (EGTA), 2,2′,2″-(1,4,7-triazonane-1,4,7-triyl)triacetic acid (NOTA), an 1,4,7,10-tetraazacyclododecane-1,4,7,10-tetraacetic acid (DOTA), carboxymethylated aspartic acid (CM-Asp), tris(carboxymethyl)-ethylenediamine (TED) and combinations thereof.

18. The method of claim 15, wherein the polyamine compound is tris(2-aminoethyl) amine (TREN).

19. The method of claim 1, wherein the adapter molecule has the formula 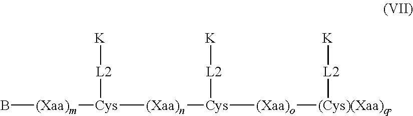
or the formula 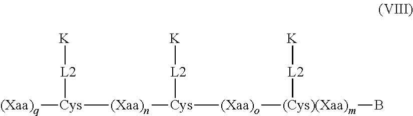
wherein Xaa is any amino acid and n, and o, have independently from each other a value of 0, 1, 2, 3 or 4, and m and q have independently from each other a value between 0 and 50 and wherein each cysteine is linked (via the sulfur (S) atom of the side chain) via an optionally present linker moiety L2 to a chelating ligand K and wherein -(Xaa)ₘ-Cys-(Xaa)ₙ-Cys-(Xaa)ₒ-(Cys)(Xaa)_{q} and (Xaa)_{q}-Cys-(Xaa)ₙ-Cys-(Xaa)ₒ-(Cys)(Xaa)ₘ, respectively are denoted by SEQ ID NO: 17).

20. The method of claim 1, wherein the adaptor molecule has the formula:
-Trp-Ser-His-Pro-Gln-Phe-Glu-Lys-Cys(C₁-Cₙ₁-NTA)-Cys(C₁-Cₙ₂-NTA)Cys(C₁-Cₙ₃-NTA)-,
wherein each of (C₁-Cₙ₁-NTA), (C₁-Cₙ₂-NTA) and (C₁-Cₙ₃-NTA) is covalently linked to the S atom of the respective cysteine residue (formula IX) or
-Trp-Ser-His-Pro-Gln-Phe-Glu-Lys-(Xaa)ₙ-Cys(C₁-Cₙ₁-NTA)-Cys(C₁-Cₙ₂-NTA)-Cys(C₁-Cₙ₃-NTA)-(Xaa)-Trp-Ser-His-Pro-Gln-Phe-Glu-Lys- (SEQ ID NO: 9) wherein Xaa is any amino acid and n is an integer from 0 to 10 (formula X, wherein each (C₁-Cₙ₁-NTA), (C₁-Cₙ₂-NTA) and (C₁-Cₙ₃-NTA) is covalently linked to the S atom of the respective cysteine residue, wherein each of n1, n2 and n3 has a value independently selected from 1, 2, 3, 4, 5, 6, 7, 8, 9, 10, 11, 12, 13, 14, 15, 16, 17, 18, 19, or 20, and wherein main chain carbon atoms of each of the C₁-Cₙ₁, C₁-Cₙ₂ or C₁-Cₙ₃ group are optionally substituted by one or more heteroatoms selected from the group consisting of N, O and S.


SEQUENCE LISTING (21 sequences)

SEQ ID NO: 1 - protein, 8 aa, Artificial Sequence
  streptavidin binding peptide
WXHPQFXX

SEQ ID NO: 2 - protein, 8 aa, Artificial Sequence
  streptavidin binding peptide
WRHPQFGG

SEQ ID NO: 3 - protein, 8 aa, Artificial Sequence
  streptavidin binding peptide
WSHPQFEK

SEQ ID NO: 4 - protein, 4 aa, Artificial Sequence
  streptavidin binding peptide
HPQF

SEQ ID NO: 5 - protein, 8 aa, Artificial Sequence
  streptavidin binding peptide
XXHPQFXX

SEQ ID NO: 6 - protein, 8 aa, Artificial Sequence
  streptavidin binding peptide
WXHPQFXX

SEQ ID NO: 7 - protein, 8 aa, Artificial Sequence
  streptavidin binding peptide
WSHPQFEK

SEQ ID NO: 8 - protein, 17 aa, Artificial Sequence
  streptavidin binding peptide
WSHPQFEKXWSHPQFEK

SEQ ID NO: 9 - protein, 21 aa, Artificial Sequence
  streptavidin binding peptide
WSHPQFEKXCCCXWSHPQFEK

SEQ ID NO: 10 - protein, 20 aa, Artificial Sequence
  streptavidin binding peptide
WSHPQFEKGGGSWSHPQFEK

SEQ ID NO: 11 - protein, 10 aa, Artificial Sequence
  Myc-tag
EQKLISEEDL

SEQ ID NO: 12 - protein, 9 aa, Artificial Sequence
  HA-tag
YPYDVPDYA

SEQ ID NO: 13 - protein, 11 aa, Artificial Sequence
  VSV-G-tag
YTDIEMNRLGK

SEQ ID NO: 14 - protein, 11 aa, Artificial Sequence
  HSV-tag
QPELAPEDPED

SEQ ID NO: 15 - protein, 14 aa, Artificial Sequence
  V5-tag
GKPIPNPLLGLDST

SEQ ID NO: 16 - protein, 8 aa, Artificial Sequence
  FLAG-tag
DYKDDDDK

SEQ ID NO: 17 - protein, 7 aa, Artificial Sequence
  adapter molecule
XCXCXCX

SEQ ID NO: 18 - protein, 15 aa, Artificial Sequence
  S-tag
KETAAAKFERQHMDS

SEQ ID NO: 19 - protein, 38 aa, Artificial Sequence
  SBP-tag
MDEKTTGWRGGHVVEGLAGELEQLRARLEHHPQGQREP

SEQ ID NO: 20 - protein, 26 aa, Artificial Sequence
  calmodulin binding peptide
KRRWKKNFIAVSAANRFKKISSSGAL

SEQ ID NO: 21 - protein, 51 aa, Artificial Sequence
  chitin binding domain
TNPGVSAWQVNTAYTAGQLVTYNGKTYKCLQPHTSLAGWEPSNVPALWQLQ